(19) United States
(12) Patent Application Publication
(10) Pub. No.: US 2011/0183367 A1
(43) Pub. Date: Jul. 28, 2011

(54) DEVICE AND METHOD FOR NON-INVASIVE MEASUREMENT OF THE INDIVIDUAL METABOLIC RATE OF A SUBSTANTIALLY SPHERICAL METABOLIZING PARTICLE

(76) Inventors: Lars Ditlev Morck Ottosen, Aarhus N. (DK); Niels B. Ramsing, Risskov (DK); Lars R. Damgaard, Brabrand (DK); Jens K. Gundersen, Viby J. (DK)

(21) Appl. No.: 12/948,301

(22) Filed: Nov. 17, 2010

Related U.S. Application Data
(62) Division of application No. 10/540,349, filed on Sep. 1, 2005, now abandoned.

(30) Foreign Application Priority Data
Dec. 23, 2002 (DK) PA200202001

(51) Int. Cl. C12Q 1/02; C12M 1/34

(52) U.S. Cl. 435/29; 435/287.1

(57) ABSTRACT

The present invention relates to methods and devices for non-invasive and non-disturbing measurements of metabolizing rates of substantially spherical metabolizing particles, such as an embryo, and to a method and device for controlling oxygen partial pressure at the level of the embryo. Furthermore, the invention relates to a method for regulating supply of metabolites to a substantially spherical metabolizing particle, as well as a method for selecting substantially spherical metabolizing particles of a predetermined quality. The invention is carried out in a device capable of establishing a diffusion gradient of metabolites between the substantially spherical metabolizing particle inside a compartment in the device and the environment outside the compartment. The metabolizing rate is determined based on information of the metabolite diffusion gradient.

### CROSS-REFERENCE TO RELATED APPLICATIONS

This application is a divisional of U.S. patent application Ser. No. 10/540,349 filed Sep. 1, 2005, which is a 371 of PCT/DK03/00935 filed Dec. 23, 2003, which claims priority of Danish Patent Application PA 2002 02001 filed Dec. 23, 2002.

### FIELD OF INVENTION

The present invention relates to methods and devices for non-invasive and non-disturbing measurements of metabolic rates for substantially spherical metabolizing particles and to a method and device for controlling metabolite concentration at the level of the particles.

### BACKGROUND OF INVENTION

Use of Embryo Transfer (ET) techniques, such as IVF (In Vitro Fertilization) and related techniques, involves in vitro culturing of the developing embryo for a period of days before re-implantation of selected embryos. Even with the ideal growth conditions, selection criteria are needed as a tool to choose the most viable embryos for re-implantation. The viability of an embryo is an important parameter in order to determine the embryos suitability for transfer. At present, there are no objective means applicable on a practical level, which can serve to assess the viability of the embryo following manipulation. In practice, embryo evaluation is limited to a more or less subjective grading based on morphological criteria.

The respiration rate of the embryo may prove a good candidate for an objective viability indicator. It has previously been demonstrated that the respiration rate of bovine, murine and human embryos (expressed as oxygen consumption) is a usable indicator of embryo viability (See Shiko et al. 2001. Oxygen consumption of single bovine embryos probed by scanning electrochemical microscopy. Anal. Chem. 73:3751-3758 or Trimarchi et al. 2000. Oxidative phosphorylation dependent and -independent oxygen consumption by individual preimplantation mouse embryos. Biology of reproduction 62: 1866-1874 or Overström E W et al. 1992. Viability and oxidative metabolism of the bovine blastocyst. Theriogenology 37(1): 269 or Magnusson C et al. 1986. Oxygen consumption by human oocytes and blastocysts grown in vitro. Human Reproduction 1: 183-184). These studies demonstrated that a certain (high) respiration rate is correlated with an improved development such as improved in vitro development (expressed by increased blastocyst frequencies) or increased pregnancy frequencies.

A number of methods for determination of embryo respiration are known. Mills and Brinster (See Mills and Brinster 1967. Oxygen consumption of preimplantation mouse embryos. Exp. Cell. Res., 47: 337-344) describe a method using the Cartesian diver technique on batches of mouse embryos, which measures the volume change of an oxygen gas bubble in direct contact with the growth medium of the embryos.

Magnusson et al. 1986 (Oxygen consumption by human oocytes and blastocytes grown in vitro. Human Reproduction 1, 183-184) and later Houghton et al. 1996 (Oxygen consumption and energy metabolism of the early mouse embryo. Molecular reproduction and development 44:476-485) describe a method which is capable of measuring oxygen consumption of individual embryos using a sensitive micro-spectrophotometric technique, where embryos are placed in small sealed chambers and the oxygen consumption is estimated as a decrease in oxygen partial pressure, monitored as an absorbance change of a substance which optical absorbance is sensitive to the presence of oxygen. Due to the extensive handling of the embryo in and out of sealed chambers, the measurements are disturbing to the embryo as well as time consuming.

Another technique has been described in which embryos are fixed on a thin capillary and oxygen concentration gradients are measured with very precisely positioned oscillating oxygen microelectrodes under the assumption of spherical diffusion (See Shiko et al., 2001. Oxygen consumption of single bovine embryos probed by scanning electrochemical microscopy. Anal. Chem. 73: 3751-3758, or Trimarchi J R, et al., 2000. Oxidative phosphorylation dependent and independent oxygen consumption by individual preimplantation mouse embryos. Biology of reproduction 62: 1866-1874). These techniques are characterized by relatively complicated experimental designs which are demanding to operate, and results in significant disturbance of the embryo. It is furthermore time consuming to perform the measurement and the presumptions for the method are demanding to fulfill.

In general, the above-mentioned studies and related studies to measure individual embryo respiration suffer from being complicated, disturbing to the embryo and time consuming, and it is therefore not very likely that such methods will be applied routinely for monitoring individual respiration rates of embryos in cultures in vitro. A need therefore still exists for a fast, simple and non-disturbing method and device for measuring individual embryo respiration rates, as a measure for the embryo viability. This need is widely expressed by researchers and practitioners of embryo transfer techniques involving in vitro culture of embryos. Overström 1996 (See Overström E W 1996, In vitro assessment of embryo viability. Theriogenology 45:3-16) compiles in a literature review the demand for a simple and objective method for determination of individual embryo respiration, as an expression of embryo viability. As the embryo in vitro techniques becomes more sophisticated, including ICSI (Intra Cytoplasmic Sperm Injection), cloning and freeze cycles, this demand is expected to become even more pronounced. Within the field of human infertility treatment, it has become necessary to focus on single embryo transfer to avoid unwanted multiple pregnancies, which are the consequence of multiple embryo transfer. Single embryo transfer, however, calls for a close viability assessment in order be able to select the best embryos and thereby increase the probability of a successful pregnancy, which again stresses the need for simple and objective viability indicators applicable on a routine level. A new method should preferably contain the following key elements as outlined by Overström 1996 (see In vitro assessment of embryo viability. Theriogenology 45:3-16 1996).

The ability to make simultaneous objective measurements of multiple individual embryos.

The sensitivity and resolution to measure an individual embryo/oocyte.

Rapid evaluation (˜30 min or less).

Viability test must be non-perturbing and ideally non-invasive.

Technically simple and user friendly.

Affordable.

In addition to the expressed need for a method and device for respiration measurements applicable on a routine level, in vitro culture of embryos suffers from an insufficient control of the oxygen partial pressures as experienced by the developing embryo. In vitro culture of embryos is often carried out in incubators with regulated atmosphere (temperature, relative humidity and gas composition). Atmospheric air contains 21% oxygen (210 hPa partial pressure), but in vivo (oviduct and uterus) oxygen tensions are considered to be around 5-10% oxygen (50-100 hPa) saturation. It is therefore not surprising that, in general, embryo development is better under a 5-10% atmosphere than under air. Lim et al. and Thompson et al. (See: Lim et al. 1999 Development of in vitro bovine embryos cultured in 5% CO₂ in air or 5% O₂, 5% CO₂ and 90% N₂. Human reproduction 7(4):558-562 or Thompson J G E et al. 1990 Effect of oxygen concentration on in vitro development of preimplantation sheep and cattle embryos. J. Reprod. Fert. 89, 573-578) and others previously demonstrated the positive effect of reduced oxygen partial pressure on the mammalian embryo development. Embryos are therefore in some cases cultured under a reduced oxygen atmosphere, e.g. 5% saturation. It is however insufficient to control the embryos exposure to oxygen by alone controlling the atmosphere above the medium. The medium is typically oxygen saturated (21%) when initiating the in vitro culture, and the equilibration time between the medium and the overlaying gas atmosphere can, depending on the in vitro growth system, be as long as 12-24 hours, such that the embryo for a significant period of the in vitro culture, will experience oxygen partial pressure significantly exceeding what at present is considered the optimal (5-10%). The final steady state partial pressure at the surface of the embryo will however be lower than that of the above atmosphere, e.g. 5%, due to the steady state oxygen partial pressure gradient from the bulk medium towards the embryo, arising as a result of the embryo respiration.

A need therefore still exists for a simple and fast (<1 h) method to regulate the oxygen partial pressure as experienced by the developing embryo during in vitro culture.

All patent and non-patent references cited in the application, or in the present application, are also hereby incorporated by reference in their entirety.

### SUMMARY OF INVENTION

The present invention relates to a device suitable for an easy and fast measurement of the metabolic rate of a substantial spherical metabolizing particle. Accordingly, the present invention relates to a device for non-invasive measurement of the individual metabolic rate of a substantially spherical metabolizing particle, which device comprises
a) at least one compartment, said compartment being defined by a diffusion barrier and capable of comprising a medium with a substantially spherical metabolizing particle, said diffusion barrier allowing metabolite transport to and/or from the substantially spherical metabolizing particle by means of diffusion, whereby a metabolite diffusion gradient is allowed to be established from the substantially spherical metabolizing particle and throughout the medium,
b) at least one detector for measuring the concentration of a metabolite inside the compartment.

The device is suitable for measuring the metabolic rate of a metabolizing particle as well as for monitoring particles and selecting particles of a specified status. Thus, the present invention further relates to a non-invasive method for determining the metabolic rate of a substantially spherical metabolizing particle, comprising
a) providing at least one device as defined above,
b) arranging a substantially spherical metabolizing particle in the medium of a compartment,
c) measuring a metabolite concentration inside the compartment obtaining a metabolite concentration measure, and
d) correlating said metabolite concentration measure to a metabolic rate of said substantially spherical metabolizing particle.

And the invention further relates to a method for regulating metabolite supply to a substantially spherical metabolizing particle during culturing, comprising
a) providing at least one device comprising a compartment with medium,
b) culturing a substantially spherical metabolizing particle in the medium of a compartment, and optionally
c) measuring a metabolite concentration inside the compartment obtaining a metabolite concentration measure, and optionally
d) correlating said metabolite concentration measure to a metabolic rate of said substantially spherical metabolizing particle and optionally
e) regulating the metabolite supply depending on the metabolite concentration measure and/or the metabolic rate of said substantially spherical metabolizing particle.

In another aspect the invention relates to a method for selecting a viable embryo comprising,
a) determining the metabolic rate of the embryo at least once during culturing, and
b) selecting the embryo having an optimal metabolic rate.

The invention is particular suitable for determining the metabolic rate for a particle in an open system communicating with the surroundings. However, the device according to the invention may also be used for determining the metabolic rate in a closed system.

Accordingly, in yet another aspect the invention relates to a non-invasive method for determining the metabolic rate of a metabolizing particle, comprising
a) providing at least one device as defined above,
b) culturing a metabolizing particle in the medium of a compartment,
c) reducing metabolite supply to the medium during at least a part of the culturing period,
d) measuring a metabolite concentration inside the compartment obtaining a metabolite concentration measure after the metabolite supply has been reduced, and
e) correlating said metabolite concentration measure to a metabolic rate of said substantially spherical metabolizing particle.

Furthermore, the invention relates to an optimized culturing device, said device a device comprises at least one compartment, said compartment being defined by a diffusion barrier and capable of comprising a medium with a substantially spherical metabolizing particle, said diffusion barrier allowing metabolite transport to and/or from the substantially spherical metabolizing particle by means of diffusion, whereby a metabolite diffusion gradient is allowed to be established from the substantially spherical metabolizing particle and throughout the medium.

In yet another aspect the invention relates to a method for culturing a particle as defined here, comprising
a) providing at least one device comprising a compartment with medium,
b) culturing a substantially spherical metabolizing particle in the medium of a compartment, and optionally
c) regulating the metabolite supply to and/or from said substantially spherical metabolizing particle.

### DESCRIPTION OF DRAWINGS

Explanation to numbers on figures: Each reference consists of two numbers in the form x.x, where the first number refers to figure number and the second number refers to a specification on each figure, but such that:

x.1 refers to: Metabolizing particle

x.2 refers to: Surrounding medium

x.3 refers to: Detector

x.4 refers to: Metabolite permeable diffusion barrier

x.5 refers to: Substantially metabolite impermeable compartment wall

x.6 refers to: Metabolite permeable layer capable of supporting metabolizing particle

x.7 refers to: Opening of compartment towards the surroundings outside the compartment

x.8 refers to: Theoretical metabolite concentration gradient

x.9 refers to: Insert in embodiment according to FIG. 1

x.10 refers to: Adjustable bottom of compartment

x.11 refers to: Concentration gradient iso-lines

x.12 refers to: CCD camera

x.13 refers to: a viscous layer to cover the medium to prevent evaporation and turbulence

x.14 refers to: insertion port (FIG. 7 only)

x.15 refers to: spacers (FIGS. 9 and 10 only)

x.16 refers to: support structure

x.17 refers to: adjustable top (FIG. 16 only)

x.18 refers to: threads

FIG. 1 is a cross section of a first embodiment of a diffusion compartment with an oxygen detector at the bottom, according to the present invention. The theoretical steady state oxygen gradient is shown in a graph next to the drawing. The permeable diffusion barrier is in this case a stagnant body of medium.

FIG. 2 is a cross section of a compartment with an insert in embodiment according to FIG. 1, to adjust the internal transverse dimension of the first embodiment.

FIG. 3 is a cross section of another embodiment of the present invention comprising a diffusion compartment with an adjustable bottom.

FIG. 4 is an example of the steady state oxygen gradient measured inside a cylindrical diffusion compartment, where an embryo is cultured at the bottom. The linear part of the gradient in FIG. 4 corresponds to a section of the solid part of the line in the theoretical graph in FIG. 1. The unit on the x-axis is hPa and the unit on y-axis is μm. The position of the opening of the compartment (X.7) in relation to the gradient is marked with the vertical line.

FIG. 5A is another embodiment of the said diffusion compartment where the diffusion compartment is completely open and the oxygen gradient is recorded in two dimensions around the embryo. 5B shows a cross section of the bottom at the level of the embryo. 5C shows a hypothetical image (top or bottom view) as seen from the CCD camera, where the expected luminescence intensity of the luminophore around each individual embryo is visualized in grey tones.

FIG. 6A is an example of the steady state oxygen gradient measured towards an embryo along the plane bottom of an open compartment as illustrated in FIG. 5. FIG. 6B is a plot to illustrate how the actual gradient fit to a theoretically ideal spherical gradient. If the plot is linear, the assumption of a spherical diffusion system is fulfilled.

FIG. 7. (Design example) Transversal section through a design formed as a pipette, with which the studied metabolizing particle is picked up from a transfer container. The plunger of the pipette is particular in that it has a gas detector. After the respiring particle has been picked up, the pipette is turned with the tip up and inserted through a port in the bottom of a media vessel. The media vessel is subsequently filled with medium). The barrel of the pipette serves as the side walls of the compartment.

FIG. 8. (Design example): Transversal section through a design, where the metabolizing particle is placed in a shallow well in a plate. The well has a metabolite permeable lid with varying thickness, and thus varying metabolite transmission capacity, that can cover the well with different sections by horizontal displacement. The diffusion barrier between the medium and the surroundings can thus be adjusted by placing different sections of the lid immediately above the well. In this figure, the medium outside the well is in the form of a droplet, but could also be in the form of a larger body.

FIG. 9. (Design example): Transversal section through a design where the metabolizing particle is placed near a detector under an impermeable disk. The disk, which constitutes the upper part of the substantially impermeable compartment wall, is supported by spacers to keep a well-defined distance to the lower part of the substantially impermeable compartment wall. The spacers are shown with a hatched line to indicate that they only occupy a small fraction of the area under the disk and do not constitute a significant barrier to diffusion. Centrally located under the disk is a shallow well in which the metabolizing particle is placed. The permeability of the permeable diffusion barrier can be adjusted by changing the height of the spacers supporting the upper wall (lid) of the substantially impermeable compartment walls.

FIG. 10. (Design example) Transversal section through a design where the respiring particle is placed in a cone-shaped impression in an impermeable plate. A detector is located near the tip of the cone, and a cone-shaped impermeable lid is placed in the impression. Spacers ensure that a well-defined distance is kept between the lid and the impression.

FIG. 11. (Design example) Transversal section through a design where the compartment consists of cavity (11.4) through an impermeable block of material (11.5) placed on an impermeable plate (11.5). The cavity is largely cylindrical (or polyhedral) and filled with media, but may hollowed out near the end facing the plate to form a receptacle for the metabolizing particle (11.1). The luminophore (11.3) are placed in the extended cavity near the bottom plate (11.5).

FIG. 12 (Design example) Depression with partly open lid (can be adjusted). The detector has the form of a flat surface under the metabolizing particle, e.g. a fluorophore sheet.

FIG. 13 (Design example) Depression with a central pore (non-adjustable).

FIG. 14 (Design example) Cube where the metabolizing particle falls into the cube and is retrieved by turning cube and letting it fall out by gravity. There are two entrances such that a water flow can be forced through the cube to flush the respiring particle out.

FIG. 15 (Design example) Bent capillary with funnel at end. The detector has the form of two circular areas on the inside of the capillary, e.g. as a layer of fluorophore. The position of the respiring particle and thus the length of the diffusive barrier can be adjusted by changing the position of the capillary on the supports, as the position will determine the position of the lowest point in the capillary to where the metabolizing particle will travel by gravity.

FIG. 16 (Design example) Adjustable bottom in a dial setup. This particular embodiment provides yet another compartment with adjustable volume, such that the permeability of the permeable diffusion barrier, in this case a stagnant body of medium, can be adjusted by changing the thickness of the layer and thus altering the permeability coefficient. The thickness of the permeable layer is reduced by turning 16.17 clockwise, whereby 16.17, by means of a thread 16.18, is moved towards the bottom of the large well containing surrounding medium 16.2. As the bottom of the compartment is fixed relative to the bottom of the larger well this results in a decrease of the compartment volume and thus in a decreased thickness of the permeable layer and therefore also in an increased permeability. The detector extends from the bottom of the compartment towards the bottom of the larger well containing surrounding medium, where it can be brought in contact with a recording unit.

FIG. 17 (Design example) Plate with depressions. This embodiment consists of a plate with several, e.g. 500-3000 μm deep conical depressions of a suitable angle 30 (such as 15 to 60 degrees), placed in yet another depression with a hydrophilic surface. The remaining part of the plate surface is hydrophobic. A drop 17.2 of a suitable volume, 10-20 μl, fills the two depressions and makes the permeable diffusion barrier. A layer of suitable oil above the drop prevents evaporation from the drop and convection inside the drop such that the body of medium for practical purposes is kept stagnant. Alternatively, the volume outside the conical depression makes the surrounding medium and is not specifically included in the permeable diffusion barrier, unless it for other reasons remains stagnant. The permeability of the diffusion barrier can be adjusted through applying conical depressions (compartments) with different angles or depths, and the permeability of a particular conically shaped compartment can be calculated according to the equations in example 4.

FIG. 18: Measuring respiration rates for mouse embryos in the setup shown in FIG. 11 and described in Example 6 (Skorstens example). Raw fluorescence data. The fluorescence intensity from the oxygen quenchable porphyrin fluorophor (Platinum (II)-octa-ethyl-porphyrin in polystyrene), in contact with the medium in the incubation chamber), was recorded using excitation light at 360 and 550 nm respectively and recording emission light at 650 nm in a Tecan Spectraflour fluorescents plate reader. Fluorescence was recorded from 0 to 500 μs after excitation.

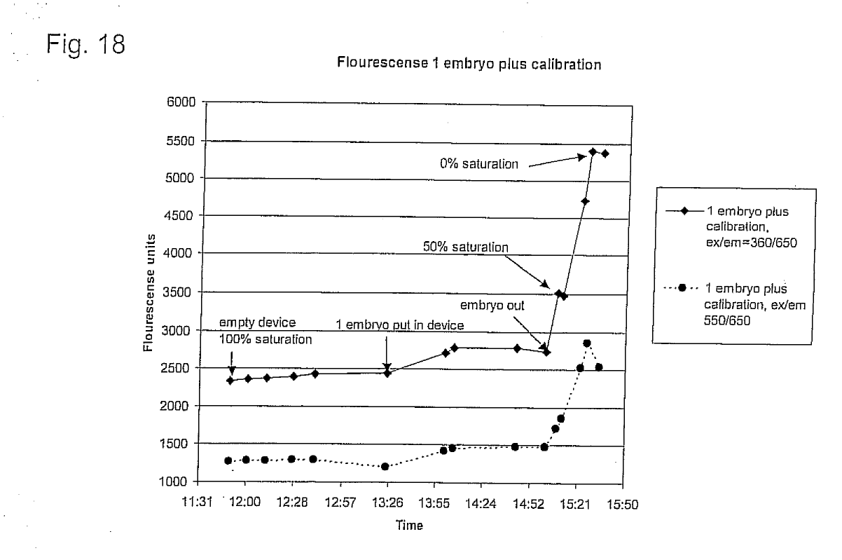

FIG. 19: Measuring respiration rates for mouse embryos in the setup shown in FIG. 11 and described in Example 6 (Skorstens example). Measured oxygen concentrations, calibrated data. Fluorescence intensities were converted to oxygen partial pressure using a modified Stem-Volmer equation, which adequately describes the response of most optrodes, according to Klimant et al 1995 (Fiber-optic oxygen microsensors, a new tool in aquatic biology. Limnol Oceanogr 40:1159-1165).

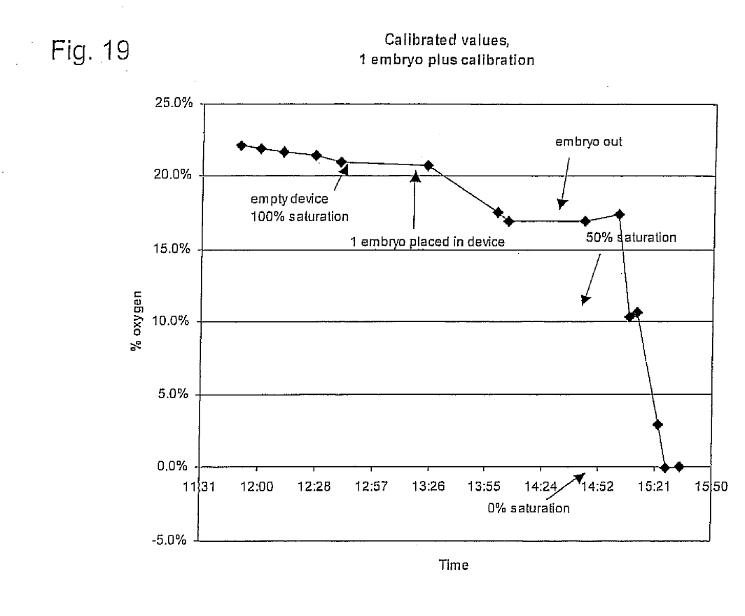

FIG. 20: Measuring respiration rates for a mouse embryo performed with oxygen microsensors as described in Example 7 using the design shown in FIG. 17.

### DEFINITIONS

Amperometric oxygen sensor: A Clarck type electrochemical sensor with a gold cathode polarized against an internal reference, where oxygen is reduced on the cathode surface. A current meter converts the resulting reduction current to a signal.

Bottom of the compartment: In the present context the term “bottom of the compartment” means the part of the compartment being located further away from any metabolite permeable opening as compared to the substantially spherical metabolizing particle. The “bottom” does not necessarily indicate a vertical position below the substantially spherical metabolizing particle, but may be the side of the compartment opposing an opening.

Bulk medium: Medium in the surroundings outside the compartment or at a distance from the metabolizing particle such that the metabolism of the particle does not influence the metabolite concentration of the bulk medium.

Diffusion: The process whereby particles of liquids, gases, or solids intermingle as the result of random molecular motions caused by thermal agitation, resulting in a net transport of dissolved substances from a region of higher to one of lower concentration.

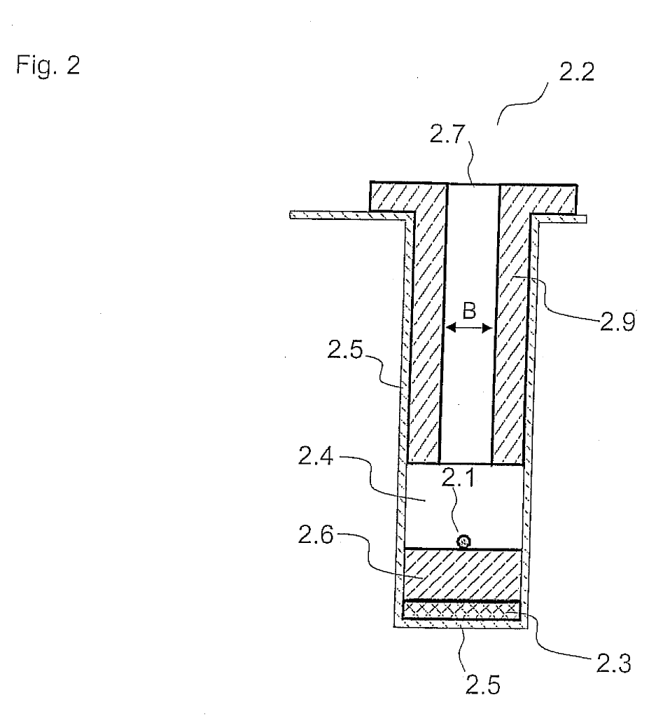

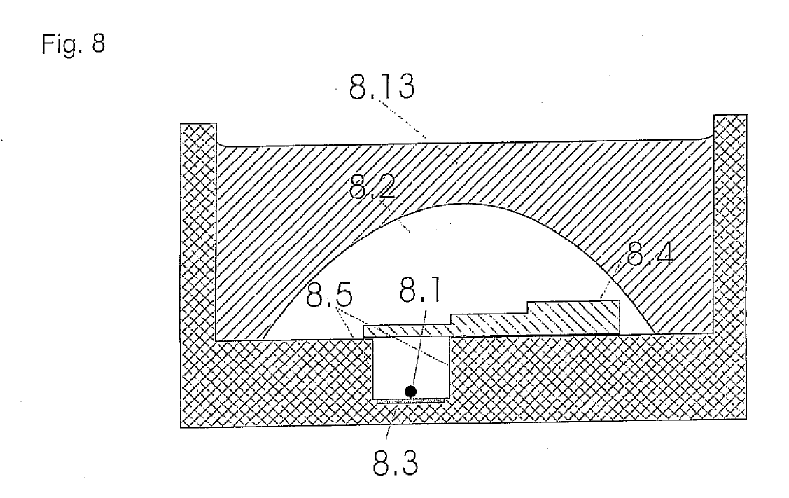

Diffusion barrier: In the present context the “diffusion barrier” means both the impermeable material which restricts the diffusive flow of metabolites to the metabolizing particle and the permeable material through which the metabolite taken by the particle passes by molecular diffusion. It may in some cases also refer to the volume and particular geometry, which the permeable material and impermeable material occupies. In a preferred embodiment the diffusion barrier consists of one or more medium filled openings bounded by impermeable walls, but it may also contain other permeable materials such as silicone or other polymer (see above). If the diffusive pathway taken by metabolites from the bulk media to the metabolizing particle passes through a constricted area with a reduced cross section and/or reduced permeability such as the insert of FIG. 2, or the lid of FIG. 8 then this region is particularly limiting for the area integrated flow. It will thus encompass the largest and sharpest metabolite concentration gradients and this part of the device is therefore often referred to as the “diffusion barrier”.

Diffusion compartment: A space or compartment of defined internal dimension with a defined opening towards an exterior environment. The liquid based material inside the diffusion compartment is stagnant, primarily due to frictional forces between the liquid and the compartment wall. The diffusion compartment is also referred to as the “compartment” in the device and method of the present invention.

Impermeable material: In the present context an “impermeable material” or “substantially impermeable material” means a material with markedly reduced permeability for the metabolite in question as compared to water, preferably the permeability is reduced to <1% for the metabolite in question as compared to water, more preferably reduced to <0.2% or <0.05%, so that the area integrated flux through this material to the metabolizing object is much lower than the flux through the permeable material (e.g. opening, permeable membrane and/or diffusion barrier). The area integrated flux through the impermeable or substantially impermeable material should be <10%, preferably <1% or most preferably <0.01% of the total area integrated flux to the metabolizing particle.

Luminescence: Production of light. In the context of the present invention the luminescence arise due to absorbance of light by a luminophore and subsequent return to the ground state after emission of light with a longer wavelength. This process is often referred to as fluorescence or phosphorescence depending on the type and lifetime of the decay.

Medium: Liquid growth substance for the embryo, such as a fluid growth substance, preferably a liquid growth substance.

Membrane inlet mass spectrometry (MIMS): A technique for measuring oxygen and other dissolved gasses, based on a tube equipped with a gas permeable membrane, connected to the inlet of a mass spectrometer. Due to a vacuum (applied by the mass spectrometer) inside the tube, gas enters the mass spectrometer through the gas permeable membrane. The concentrations of selected gasses are subsequently determined by the mass spectrometer.

Metabolite. In the present context the term “metabolite” means a compound that is either taken up or released by the metabolizing particle. Examples of metabolites include oxygen, carbon dioxide, amino acids, glucose, ions, such as Ca⁺⁺ ions and H₃O⁺ ions.

Metabolic rate: The rate at which the metabolite in question is consumed or released by the metabolizing particle. The metabolic rate is dependent on both the metabolite in question and on the level of activity of the organism.

Metabolising. In the present context the term “metabolising” refer to the process of taking up or releasing metabolite. A preferred metabolite which is being metabolised is oxygen which is taken up and consumed by respiration.

Metabolite permeable opening: In the present context a “metabolite permeable opening” in a compartment may be used to indicate both a free opening (i.e. containing nothing but medium) and a covered opening. The latter is the case where the opening is covered with a permeable material such as a membrane, (e.g. a silicone layer) to constitute a diffusion barrier that is more permeable than the other walls of the compartment.

Metabolizing particle: in the present context the term “metabolizing particle” means a particle taking up or releasing metabolites during a period of time. A preferred type of metabolizing particle is a respiring particle which consumes oxygen by respiration. The metabolizing particle is preferably a cell or a group of cells, however the metabolizing particle may also be a synthetic particle consuming oxygen.

Microspectrophotometric technique: A technique for measuring oxygen based on an increase or decrease in absorbance at 435 nm, reflecting dissociation of oxy-hemoglobin due to a decrease or increase in oxygen partial pressure. Other oxygen binding molecules with other absorption characteristics may be used.

Noninvasive method: A method, which without any destructive disturbance, or without requiring insertion of an instrument or device through the skin or body orifice can measure a parameter related to a body of interest.

Optical oxygen sensing: A measuring principle based on the ability of oxygen to act as a dynamic luminescence quencher of a luminophore. The luminophore is excited by defined wavelengths, and luminescence is emitted by the luminescent indicator as a function of oxygen concentration. This process is often referred to as fluorescence or phosphorescence. In the presence of oxygen the intensity and the decay time of the luminescence decreases in a predictable way due to the quenching process. Optical oxygen sensing in two dimensions can be based on luminescence lifetime imaging, which in some cases is advantageous over luminescence intensity imaging.

Oxygen partial pressure: The pressure that oxygen as a single component would exert. The total gas pressure is the sum of individual gas pressures. Under normal atmospheric conditions the total actual gas pressure will be close to 1 atm or 1000 hPa. Atmospheric oxygen partial pressure is approximately 21% or 210 hPa. The oxygen concentration C is equal to the oxygen partial pressure P multiplied by the oxygen solubility S, (C=PS), where the solubility S is a function of temperature, salinity and total gas pressure.

Respiration rate: Most living organisms, including developing embryos, consume oxygen in their energy metabolism, by a process called respiration. The oxygen consumption rate of a respiring organism is also named the respiration rate. The respiration rate of human embryos has previously been determined to be in the range 0.34-0.53 nl O₂ embryo⁻¹ h⁻¹, but embryo respiration rates can vary considerably during the development from oocyte over morula to the blastocyst stage (See Magnusson C et al. 1986. Oxygen consumption by human oocytes and blastocysts grown in vitro. Human Reproduction 1: 183-184). Bovine embryos will typically have respiration rates in the range from 1-8 nl O₂ embryo⁻¹ h⁻¹.

Response time: The time from initiating a measurement until a response or signal adequate for the measurement is obtained, and the measurement can be considered successful.

Stagnant liquid: A liquid without any flow, turbulence or movement. Transport of dissolved substances primarily takes place by diffusion.

Steady state: A situation where consumption and transport are in equilibrium such that gas partial pressure, or concentration gradients of dissolved substances, are stable and no partial pressure change or concentration change takes place over time.

Substantially spherical metabolizing particle: In the present context the term “a substantially spherical metabolizing particle” means a metabolizing particle or a group of metabolizing particles, wherein the group is arranged to form a substantial sphere or ellipsoid or box shaped object, such as a group of cells, for example a multi-cell embryo.

### DETAILED DESCRIPTION OF THE INVENTION

The present invention relates to establishing the metabolisation rate of a substantially spherical metabolizing particle. The metabolic rate is preferably established non-invasively in order not to disturb the particle. The invention is based on the finding that the rate of metabolisation may be determined fast and non-invasively by measuring the concentration of a predetermined metabolite in a small volume of the environment of the particle if the environment is constructed to allow only diffusion of said metabolite to or from the particle. By such a construction a diffusion gradient of the predetermined metabolite develops in the environment and by measuring the concentration of the predetermined metabolite at only one position of the diffusion gradient knowing the concentration outside the environment it is possible to calculate the metabolite concentration at the position of the particle and thereby determine the metabolic rate of the particle.

The present invention relates to substantially spherical metabolizing particles. The metabolizing particles of interest to the present invention include a prokaryotic or eukaryotic cell or a group of such cells, however the metabolizing particle may also be a synthetic particle consuming oxygen. A preferred type of particles include an embryo, group of cells, such as cancer cell(s), stern cells, embryonic stem cells, a small multicellular organism at a life stage with a relevant size and metabolic rate (e.g. eggs, embryo or tissue samples of some of the larger organisms) such as Caenorhabditis elegans, Dictyostelium discoideum, Drosophila melanogaster, Xenopus laevis, Arabidopsis thaliana, Danio reri, Chlamydomonas reinhardtii, Aplysia californica. A most preferred particle includes mammalian embryos such as human, bovine or murine embryos.

The metabolites taken up by such particles or released by them are replenished or removed by molecular diffusion as outlined in Example 4. The devices of the present invention comprise devices with a compartment in which the substantially spherical metabolizing particle is placed. The compartment consists of permeable and impermeable material arranged around the metabolizing particle to restrict and reduce the diffusive flux of metabolites to and from the particle. If the substantially spherical metabolizing particle is arranged in an environment wherein replenishment and removal of metabolites is made unhindered by spherical diffusion effective with moderate metabolic rates, then the concentration of these metabolites are only marginally affected for a very small volume in close proximity to the respiring particle. However, if the particle is placed in a compartment, which limits the diffusive re-supply or removal of metabolites, then measurable changes in the concentration inside the compartment of these metabolites can be detected. The devices comprising the present invention accomplish this by restricting the volume through which the metabolites can pass by molecular diffusion by impermeable (or substantially impermeable) surfaces. These surfaces (or walls) does not entirely surround the metabolizing particle, but leaves a permeable opening, through which the metabolite passes by diffusion. The permeable opening(s) may be filled with medium or another permeable material. The spatial arrangement of permeable and impermeable material around the metabolizing particle constitutes the diffusion barrier. It serves three purposes: 1) to restrict the flux of metabolites so that a local deviation from the bulk concentration can be measured with a detector. 2) it enables determination of the metabolic rate for the metabolizing particle based on the magnitude of the deviation. 3) it restricts or eliminates transport of metabolite to the metabolizing particle by turbulent flow. The latter purpose of the diffusion barrier is usually accomplished by confining the medium between surfaces that are positioned so close to each other that the liquid cannot mix by turbulent flow between the surfaces. Many different examples of possible designs for devices and diffusion barriers are presented in example 5. The theoretical concentration gradients arising from different designs are shown in example 4. Experimental data arising from compartments with a cylindrical depression are presented in example 1 and 6 and with embryo laying on an impermeable surface with depressions in example 7 or without in example 3.

The theory of diffusion is presented in example 4, based on the following literature: Crank, J. 1997. The Mathematics of Diffusion. Clarendon Press.

If the nature of the gradient in the compartment, caused by the metabolisation by the substantially spherical metabolizing particle, cannot be described fully, or the internal dimensions of the compartment are not well defined, the device can be calibrated by using artificial substantially spherical metabolizing particles with a known metabolite uptake and/or release. Artificial substantially spherical metabolizing particles for calibration can be small spherical particles with the diameter of the relevant substantially spherical metabolizing particle, for example artificial embryos of the dimensions of 50-200 μm made of an oxygen consuming material (antioxidant), like vitamin C, E, A, carotenoids, selenium, titanium chloride, dithionite, ferrous sulfides, embedded in a stable auxiliary compound like starch, or coated onto inert spherical bodies like glass beads.

In case the metabolite concentration gradient inside the compartment is not in steady state and still develops, which may be the case shortly after the substantially spherical metabolizing particle is placed in the compartment, the metabolic rate may still be determined by investigating the change of the metabolite concentration gradient inside the compartment per time unit. The steady state gradient can in other words be modeled mathematically from a series of non steady state gradients over time.

### Metabolites

The metabolites measured according to the present invention may be any metabolites relevant to be either taken up by the substantially spherical metabolizing particle or released from said particle. Examples of metabolites are as described above under definitions. In one embodiment the metabolite is a gas, such as oxygen that may be detected by several methods as described below, or the metabolite is carbon dioxide, detection methods of which are also described below.

Thus, in a preferred embodiment the present invention relates to determination of the respiration rate of the substantially spherical metabolizing particle by measuring the gas partial pressure of oxygen and/or carbon dioxide.

### Compartment

As described above the present invention is based on the establishment of a diffusion gradient for the metabolite to be measured, i.e. that the physical conditions around the substantially spherical metabolizing particle allows a diffusion gradient to be established, at least during the period of time relevant for measuring the metabolic rate.

The substantially spherical metabolizing particle is placed and/or cultured in a compartment with predefined dimensions. The compartment preferably comprises medium comprising the relevant metabolites for the substantially spherical metabolizing particle. Furthermore, it is preferred that the compartment is in communication with the outside of the compartment allowing metabolites to enter the compartment and into the medium by way of diffusion. Thereby it is possible to use the compartment for culturing the substantially spherical metabolizing particle for a longer period of time without having to move the substantially spherical metabolizing particle when determining the metabolizing rate. However, it is within the scope of the present invention that the substantially spherical metabolizing particle is moved to the compartment when determining the metabolizing rate, and subsequently removed from the compartment.

In order to establish the conditions for allowing a diffusion gradient to be established, the compartment may be defined by a diffusion barrier and be capable of comprising a medium, said diffusion barrier allowing metabolite transport to and/or from the substantially spherical metabolizing particle by means of diffusion, whereby a metabolite diffusion gradient is allowed to be established from the substantially spherical metabolizing particle and throughout the medium.

The compartment establishes a local environment for the substantially spherical metabolizing particle allowing at least one metabolite to be transported to and/or from the substantially spherical metabolizing particle by diffusion only.

The medium inside the compartment surrounding the substantially spherical metabolizing particle should preferably be kept stagnant, such that transport of substances dissolved in the medium can alone take place by diffusion. Bulk medium outside the compartment does not have to be stagnant. Stagnant is as defined above.

Furthermore, the compartment should be designed so that the medium inside is kept stagnant, and furthermore so that the transport of the predetermined metabolite to the compartment is controlled in relation to the substantially spherical metabolizing particle, the metabolic rate of which is to be determined.

### Stagnant

The importance of the stagnant medium may be explained in relation to the respiration rate of an embryo: When the embryo is in the stagnant medium inside the compartment, the oxygen partial pressure close to the embryo will, due to the oxygen consumption of the embryo, be reduced compared to the oxygen partial pressure outside the compartment. In a steady state situation, the supply of oxygen equals the consumption and the oxygen partial pressure gradient towards the embryo will be stable. The steepness of the gradient from the opening of the diffusion space or at a distance from the embryo, towards the embryo, is thus a measure of the embryo oxygen consumption (respiration). The respiration rate of the embryo is measured by determining the oxygen partial pressure or concentration at a position inside the compartment. One measurement will be sufficient for determining the respiration rate under the above described conditions.

### Compartment Design and Materials

The compartment may be designed in several ways, examples of which are discussed below.

Two different principles of compartments are discussed herein below, however any compartment type capable of allowing the diffusion gradient(s) to be established fall within the scope of the present invention.

The two different principles are:
A compartment made from a wall surrounding a space
A compartment made up by and filled with a viscous material allowing a controlled diffusion of at least one metabolite.

Thus, with respect to the first principle the compartment may be defined by at least one wall constituting the outer borders of the compartment and capable of holding medium as well as the substantially spherical metabolizing particle. The wall is preferably impermeable for the metabolite to be measured. In case a polymer or a copolymer is chosen to constitute the material providing a substantially impermeable diffusion barrier, it should be characterized by a low permeability relative to the medium filling the compartment. If the wall is permeable it is of importance that the wall material is characterized by a low permeability relative to the medium filling the compartment.

When the wall as such is substantially impermeable for the metabolite to be measured, the wall must comprise at least one opening allowing transport of said metabolite to the substantially spherical metabolizing particle. Such an opening may be fully open to the surrounding environment or it may be partially or fully covered by a membrane, wherein said membrane allows transport of the metabolite to and/or from the inside of the compartment.

The barrier material (impermeable part) of the diffusion sphere surrounding the metabolising object should possess the ability to restrict the passage of metabolites or materials in general through their boundaries. Accordingly, the compartment wall may be made by any suitable material possessing the ability of restricting the passage of the metabolite through their boundaries. Plastics, composites, coatings, laminates, fabrics, metals, glass, ceramics, polymers such as acetal resins, acrylic resins, cellulosic plastics, fluoroplastics, ionomers, parylenes, polyamides, polyamide nanocomposites, polycarbonates, polyesters, polyimide, polyolefins, polyphenyl sulfides, polysulfones, styrenic resins, vinyl resins, plastic alloys, multiplayer polymers, epoxy resins, olefins thermoplastic elastomers, polyether block amides, polybutadiene thermoplastic elastomers, styrenic thermoplastic elastomers, vinyl thermoplastic elastomers, rubber materials such as butadiene rubber, butyl rubber, bromobutyl rubber, chlorobutyl rubber, polyisobutylene rubber, chlorosulfonated polyethylene rubber, epichlorohydrin rubber, ethylene-propylene rubber, fluoroelastomers, natural rubbers, neoprene rubbers, nitrile rubbers, polysulfide rubbers, polyurethane rubbers, silicone rubbers, styrene-butadiene rubbers, are examples of materials that may be used to achieve a substantially impermeable barrier layer.

The permeability of a materiel is the proportionality constant in the general equation for mass transport of a penetrant across a barrier.

$$Q = \frac{Δ mgas}{Δ t} = P \frac{A Δ p}{𝓁}$$

Where P is the permeability of the material/barrier, Q=Δmgas/Δt is the area integrated flux i.e. the transmission rate, A is the area, l is the thickness and Δp is the partial pressure difference across the barrier. P has the dimensions

[P]=(amount of permeant*barrier thickness)/(area*time*pressure gradient)

The term permeability as defined above are most commonly used for gases, whereas the term most commonly used for other dissolved metabolites is diffusivity (see example 4). In this case

$$Q = J \cdot F = D \frac{A Δ C}{𝓁}$$

Diffusive transport of gases may be described by either set of equations as The permeability P is the product of the diffusion coefficient D and the solubility S (i.e. P═S*D), where the solubility is defined as the ratio between concentration and partial pressure (i.e. S═C/p). The common units for Diffusion coefficients are cm²/s.

Both permeability and diffusion coefficients are affected by temperature. As a first approximation both increase with temperature roughly following the classical Arrhenius relationship.

Stagnant aqueous media has a permeability for oxygen of approximately 6700 cm³ mm/(m² day atm) at 37° C.

In case of the metabolite oxygen, an impermeable material may be defined as a material having a permeability of at most 40 cm³ mm/m² day atm 23° C., such as at most 35 cm³ mm/m² day atm 23° C., such as at most 30 cm³ mm/m² day atm 23° C., such as at most 25 cm³ mm/m² day atm 23° C., such as at most 20 cm³ mm/m² day atm 23° C., such as at most 15 cm³ mm/m² day atm 23° C., such as at most 10 cm³ mm/m² day atm 23° C., such as at most 5 cm³ mm/m² day atm 23° C., such as at most 2 cm³ mm/m² day atm 23° C., such as at most 1 cm³ mm/m² day atm 23° C.

Examples of oxygen permeability for selected plastics/polymers are:

Styrene-Acrylonitrile Copolymeres SAN (P 15-40 cm³ mm/m² day atm at 24° C.)
Acrylonitrile-Butadiene-Styrene Copolymeres ABS (P=39.3 cm³ mm/m² day atm at 25° C.)

### Polyvinyl Chlorides

Polybutylene Terephtalate PBT (P=15.5 cm³ mm/m² day atm)
Polyphenylene Sulfides PPS (P=11.8 cm³ mm/m² day atm)
Polyimide (P=10 cm³ mm/m² day atm),
Polycyclohexylenedimethylene Ethylene Terephtalate PETG (P=9.97 cm³ mm/m² day atm at 22.8° C.)
Polyvinylidene Fluoride PVDF (P=1.96 cm³ mm/m² day atm),
Polyethylene Terephtalate PET (P=2.4 cm³ mm/m² day atm),
Polyethylene Naphtalate PEN (P=0.525 cm³ mm/m² day atm),
Nylons/polyamides (P=0.4-1.5 cm³ mm/m² day atm),
Liquid Crystal Polymers LCP (P=0.037 cm³ mm/m² day atm at 23° C.)
Ethylene-Vinyl Alcohol Copolymers EVOH barrier layers (e.g. Capran Oxyshield OB, P=0.0021-24 cm³ mm/m² day atm)
Acrylonitrile-Methyl Acrylate Copolymer AMA (P=0.08-0.64 cm³ mm/m² day atm)

The opening(s) into the compartment could be covered by a membrane made of a metabolite permeable material, whereby the membrane constitutes a controlled diffusion barrier. (See design L in Example 5, FIG. 8)

In another embodiment the whole compartment wall could be made of a metabolite permeable material, the only provision being that the wall material is characterized by a lower permeability relative to the medium filling the compartment. Thereby the compartment wall constitutes a controlled diffusion barrier.

Both the permeable membrane and the permeable wall could have a membrane- or film-like structure or another structure, allowing a controlled significant transport of metabolite, such as oxygen to and/or from the metabolizing particle. In case of the metabolite oxygen, permeable material allowing a controlled diffusion barrier, the permeability is preferably at least 50 cm³ mm/m² day atm 23° C., such as at least 60 cm³ mm/m² day atm 23° C., such as at least 750 cm³ mm/m² day atm 23° C., such as at least 80 cm³ mm/m² day atm 23° C., such as at least 90 cm³ mm/m² day atm 23° C.

Examples of suitable materials for an oxygen permeable material are:

Polysulfones (P=90.5 cm³ mm/m² day atm 23° C.)
Polypropylenes PP (P=59-102 cm³ mm/m² day atm at 23° C.)
Cyclic Olefin Copolymer COC (P=71 cm³ mm/m² day atm)
Polycarbonates (P=90-120 cm³ mm/m² day atm)
Polystyrenes PS (P=117-157 cm³ mm/m² day atm),
Polyethylenes PE (P (ULDPE)=280, P (LDPE)=102-188, P (HDPE)=35-110, P (LLDPE)=98-274 cm³ mm/m² day atm)
Ethylene-Acrylic Acid Copolymeres EAA (P=178-550 cm³ mm/m² day atm)
Polytetrafluoroethylenes, PTFE Teflon (P=223 cm³ mm/m² day atm at 25° C.)
Ethylene-Vinyl Acetate Copolymeres EVA (P=177-210 cm³ mm/m² day atm)

An example of a very permeable polymer could be

Silicone (P=17280 cm³ mm/m² day atm)

These mentioned materials only constitute examples and other materials with suitable permeability and characteristics may be chosen to obtain a desired combination of diffusion barriers around the said respiring particles. Further examples of relevant polymers are listed with permeability coefficients in: Liesl K. Massey: Permeability properties of plastics and Elastomeres, a guide to packaging and barrier materials 2^{nd} edition, P 57-507). Even further examples are listed in: Brandrup & Immergut, Polymer Handbook 4^{th} edition).

In one specific embodiment the compartment is made from a gas impermeable material having at least one opening, which opening is gas permeable. The opening could be covered by a gas permeable membrane. In one particular embodiment the side walls and the bottom are made of a gas impermeable material. The compartment comprising the substantially spherical metabolizing particle in a suitable growth medium is in open connection with an atmosphere of known gaseous composition, and controlled temperature and humidity, directly via the opening or through a larger volume of medium outside the compartment. Oxygen and other dissolved substances are supplied to the substantially spherical metabolizing particle directly from the atmosphere or via the larger volume of medium in equilibrium with the atmosphere, through the defined diffusion compartment by diffusion through the stagnant medium inside the compartment. The oxygen partial pressure outside the compartment will in both cases be known. Either the composition of the atmosphere is known or the bulk medium will be in equilibrium with the atmosphere of known composition.

In another embodiment the compartment is defined by a culture medium of either high viscosity or surrounded by a medium of higher viscosity and/or polarity.

Commercially available culture media (from e.g. Sigma, Medicult, In vitro Life, Nidacon) has normally a diffusion coefficient approximately like water of same salinity. Such culture medium may be changed by either suspending impermeable particles or objects in the medium or increasing the viscosity of the medium.

Culture medias may be changed by suspending impermeable particles or objects, in order to reduce porosity and thus the diffusion coefficient, D. When gasses or other metabolites diffuse in a mixture of a liquid and impermeable particles, Dₘᵢₓₜᵤᵣₑ=D_{liquid}*porosity.

Culture medium may also be changed by increasing viscosity, to achieve a medium with a high viscosity and a substantially reduced diffusion coefficient. Such a medium may be arise from addition of essentially inert organic solutes such as dextran, glycerol, sugars, carbohydrates, proteins, organic polymers or inorganic salts.

It is also possible to change the viscosity without substantially affecting the diffusion coefficient by addition of organic polymers such as starch, agarose or other gelling reagents. This may be of value to reduce turbulent mixing in large free liquid spaces.

Furthermore, culture medium may be enclosed for example by overlying oil, such as paraffin oil or silicon oil or other medical oil, where the oil constitutes a like or a different diffusion barrier compared to an equal body of culture medium. Both solubility and transport coefficients for turbulent and diffusive flow may differ between oil and in water.

### Compartment Shape

The compartment may in principle exhibit every suitable shape for establishing a diffusion gradient for the metabolite(s) in question. However, the shape of the compartment should preferably also facilitate the handling of the substantially spherical metabolizing particle, in particular in relation to insertion and withdrawal of the substantially spherical metabolizing particle. In the present context the shape refers to the inner dimensions of the compartment. The outer dimensions of the compartment may attain any practical shape.

Accordingly, the inner shape of the compartment may be selected from the group of a cylinder, a polyhedron, a cone, a hemisphere, or a combination thereof. In a preferred embodiment the shape is a cylinder, a cone, a combination of two cylinders or a combination of a cone and a cylinder. Examples are shown in the Drawings. More preferably the shape is a cylinder.

### Compartment Dimensions

The compartment is dimensioned to allow the establishment of the diffusion gradient as discussed above. In this respect the dimensions of the compartment relative to the uptake and/or release of metabolite of the substantially spherical metabolizing particle is important. Since the uptake and/or release of a metabolite of a given substantially spherical metabolizing particle often is depending on the size of the substantially spherical metabolizing particle, the dimensions of the compartment in relation to the size of the substantially spherical metabolizing particle is relevant.

In the following the dimensions is discussed in relation to a substantially cylindrical compartment and a substantially spherical metabolizing particle having the size of a mammalian embryo, i.e. about a diameter between 30-400 μm dependent on the developmental stage and species. For other substantially spherical metabolizing particles the person skilled in the art may calculate the suitable dimensions accordingly.

Typically the transverse dimensions of the compartment is less than 2.5 mm, particularly less than 1.5 mm, more particularly less than 500 μm, such as less than 250 μm.

The longitudinal dimension of the compartment is in one embodiment between 2 to 25 mm, particularly between 3 to 15 mm. The longitudinal dimension is usually the vertical height of the medium constituting the diffusion barrier. In generalized terms it is the distance perpendicular to the diffusion gradient from metabolizing particle to the mull medium.

The dimensions may be the dimensions of the compartment as such, or it may be provided by inserting one or more inserts in a standard compartment thereby facilitating the use of the same type of compartments for measuring metabolic rate of several different types of substantially spherical metabolizing particles.

In one embodiment the compartment has at least one insert for the adjustment of the transverse dimensions of the compartment. In a preferred embodiment the inner transverse dimensions of a cylindrical insert is as defined above, such as less than 2.5 mm, particularly less than 1.5 mm, more particularly less than 500 μm, such as less than 300 μm.

In another embodiment, the dimensions may also be adjusted by providing the compartment with an adjustable bottom, such as for example wherein the compartment is formed with a plunger-like bottom. Thereby the dimensions of the compartment may be both increased and decreased.

The adjustable bottom may be used in combination with insertion of one or more inserts as is appropriate in the specific situation.

The functional compartment dimensions may also be changed by changing the volume of medium in the compartment. The medium level in the compartment can be varied in a controlled way by adding or removing a defined quantity of medium. The functional principle of this relates to increasing or decreasing the distance in the stagnant medium through which the metabolite oxygen has to diffuse, corresponding to altering the dimensions of the effective diffusion compartment and thus controlling the transport of metabolite from the outside of the compartment of constant composition, to the substantially spherical metabolizing particle. The metabolic rate of the substantially spherical metabolizing particle can be determined with the option of adjusting the medium level and thus the metabolite concentration as experienced by the substantially spherical metabolizing particle to a desired level, at any metabolic rate.

### Metabolite Permeable Layer

In one embodiment the substantially spherical metabolizing particle is arranged in the compartment on a layer of metabolite permeable layer. Thereby the substantially spherical metabolizing particle is supplied with metabolite from all sides leading to more optimal conditions. Another advantage of the metabolite permeable layer, is that it may facilitate the measurement of the metabolite concentration as discussed below. The metabolite permeable layer is preferably arranged in the bottom of the at least one compartment, wherein the bottom is as defined above.

The metabolite permeable layer may be produced from any material permeable to the metabolite in question, as discussed above for the metabolite permeable membrane. In particular the metabolite permeable layer may be produced from a material comprising silicone, Teflon fluoropolymers, plastic compounds such as polyethylene, polypropylene or neoprene.

In another embodiment the metabolite permeable layer is produced from a material comprising permeable matrixes or porous material such as glass, ceramics, minerals, glass or mineral fibers, or precious metal such as gold or platinum.

In yet another embodiment the metabolite permeable layer is produced from a material comprising silicone.

The thickness of the metabolite permeable layer is dimensioned to the purposes it should serve, as described above. In a preferred embodiment the thickness of the metabolite permeable layer is preferably at least twice the diameter of the substantially spherical metabolizing particle, such as at least 100 μm, particularly at least 300 μm, and more particularly at least 900 μm.

### Detection Methods

The metabolite concentration inside the compartment is preferably measured by a non-invasive method. The method is appropriately chosen depending on the metabolite in question.

In one embodiment the metabolite is oxygen consumed by the substantially spherical metabolizing particle. Oxygen detection can be based on optical sensing (see definitions) with immobilized luminophore, optical sensing with luminophores dissolved in the medium, microspectrophotometric techniques, electrochemically based oxygen sensors including Clack type oxygen sensors, MIMS technology (membrane inlet mass spectrometry) or any other means of detection conceivable by a person skilled in that art. In a particular embodiment of the invention the oxygen partial pressure or concentration is determined using an immobilized luminophore layer and recording the luminescence with a luminescence reader or a camera such as a CCD-camera, or a photomultiplier tube.

Optical oxygen sensors are mainly based on the principle of luminescence quenching. The lower the oxygen concentration the weaker the quenching becomes and an increased luminescence is observed. Based on a modified Stern-Volmer equation the following is found

$$C = \frac{{I}_{0} - I}{{K}_{SV} ( I - {I}_{0} α )} \qquad ( 1 )$$

where α is the nonquenchable fraction of the luminescence, I₀ is the luminescence intensity in the absence of oxygen, and K_{SV} is a constant expressing the quenching efficiency of the immobilized luminiphore (Stem and Volmer 1919, Klimant et al. 1995). The concentration can be calculated based on a simple three-point calibration.

An alternative optical sensing principle has been developed for luminophores with long phosphorescence lifetimes. As the oxygen concentration decreases in the environment around the luminophore, the phosphorescence lifetime (after a single flash of light) lengthens in a systematic manner.

The oxygen dependence of phosphorescence for this type of sensors is described by the Stem-Volmer relationship

τ₀/τ=1+k_{q}·τ₀·p_{O2} (2)

where τ₀ and τ are the phosphorescence lifetimes in the absence of oxygen and at an oxygen partial pressure of ·p_{O2}, respectively, and k_{q} (the quenching constant) is a second-order rate constant that is related to the frequency of collisions between oxygen and the excited triplet state of the porphyrin and the probability of energy transfer when collisions occur. To calculate the oxygen partial pressure·p_{O2}, the quenching constant and the lifetime in the absence of oxygen must be measured.

In contrast to more commonly used intensity-based systems, the measurement of luminescence lifetime provides certain advantages, such as insensitivity to photo bleaching, uneven distribution or leaching of the dye, or changes in the intensity of excitation light. This facilitates the use of simple optical systems or optical fibres. A new family of oxygen-sensitive dyes, the porphyrin-ketones, has been introduced, which exhibits favorable spectral properties and decay times in the order of tens and hundreds of microseconds. This allows the use of simple optoelectronic circuitry and low-cost processing electronics.

Recently, a new approach for high spatial resolution studies was developed based on the use of planar optical sensor foils for oxygen in combination with imaging techniques. Here, the sensor foil can be mounted on the inside of a transparent sample container, and by monitoring the sensor foil from outside with a charge-coupled device (CCD) camera, changes in the oxygen-dependent luminescence of the sensor foil can be monitored and used for measuring the two-dimensional oxygen distribution in the sample. These foils can be used for both intensity and lifetime based measurements. They can be used as internal detectors in the novel devices described in this document. Examples of oxygen luminophores are Methalorganic dyes, such as Ruthenium (II) polypyridyl complexes, Ruthenium (II) bipyridyl complexes, Ruthenium (II) diimin complexes, Porhyrin complexes, Bis(Histidinato)cobalt(II), Platinum 1,2 enedithiolates. Preferably the oxygen lumoniphores can be made of Ruthenium(II)-tris-4,7-diphenyl-1,10-phenatroline per chlorate (Rudpp) immobilised in a polystyrene matrix, Ruthenium (II) tris-1,7-diphenyl-1,10-phenanthroline chloride, Ruthenium(II)-tris(bipyridyl) complex, Tris (2,2′-bipyridyl di-chloro-ruthenium) hexa-hydrate, Ru(bpy), Platinum (II)-octa-ethyl-porphyrin in polystyrene, Platinum (II)-octa-ethyl-porphyrin in poly(methyl-methacrylate), Platinum (II)-octa-ethyl-keto-porphyrin in polystyrene, Platinum (II)-octa-ethyl-keto-porphyrin, Palladium (II)-octa-ethyl-porphyrin in polystyrene.

Further information about the use of optical oxygen sensors and sensors for other metabolites such as glucose, pH and carbon dioxide can be found in the literature listed below.

Thus, in one embodiment, the oxygen detector can be electrochemical or any other detection principle for oxygen.

The oxygen concentration determination is in a particular embodiment performed in the bottom of the compartment, and in another embodiment the means for oxygen determination is placed at the bottom of the compartment underneath a metabolite permeable layer and the at least one substantially spherical metabolizing particle is resting on the gas permeable layer, so that the metabolite permeable layer is placed between the substantially spherical metabolizing particle and the metabolite detector.

In one embodiment the oxygen partial pressure is determined with a Clark type electrochemical oxygen micro sensor with a tip diameter not exceeding the transverse diameter of the compartment, placed at the bottom of the compartment with the sensor tip penetrating the oxygen impermeable bottom wall of the compartment. The sensor tip is separated from the substantially spherical metabolizing particle by an oxygen permeable layer. The oxygen sensor should be of a design such that the analyte (oxygen) consumption of the sensor does not exceed a negligible fraction, such as 1%, of the substantially spherical metabolizing particle respiration rate, such that the oxygen partial pressure inside the compartment gradient is not disturbed by the measuring activity of the said sensor.

In another embodiment the Clark type oxygen sensor is replaced by a MIMS fiber penetrating the oxygen impermeable bottom wall of the compartment. The MIMS fiber tip is separated from the embryo by an gas permeable layer. The MIMS fiber should be of a such design that the analyte (any gas which can migrate through the MIMS fiber membrane and is detectable on a mass spectrometer) consumption of the sensor does not exceed a negligible fraction, such as 1%, of the substantially spherical metabolizing particle consumption or production rate, such that the gradient of a particular gas inside the compartment gradient is not disturbed by the measuring activity of the said MIMS fiber.

In yet another embodiment, the oxygen partial pressure gradient inside the compartment is determined by adding oxyhaemoglobin, or another molecule with an oxygen dependant absorption characteristic, to the growth medium and measuring the absorbance gradient at 435 nm, or another suitable wavelength, through transparent sidewalls of the compartment, and thereby determining the oxygen distribution in the compartment.

Other metabolites may be measured by using luminescent indicators for these metabolites, such as luminescence indicators for carbon dioxide, Ca²⁺, and glucose.

Furthermore, pH may be measured at a given position in the compartment indicating the concentration of the metabolite in the compartment.

### Device

The device according to the present invention comprises at least one compartment as described above. In a preferred embodiment the device comprises more than one compartment, such as at least two compartments, such as at least 4 compartments, such as at least 6 compartments, such as at least 8 compartments, such as at least 12 compartments, such as at least 24 compartments, such as at least 48 compartments, such as at least 96 compartments. Thereby, the metabolic rate of more the one substantially spherical metabolizing particle can easily be determined, each compartment comprising one substantially spherical metabolizing particle.

It is preferred that the compartment is suitable for culturing the substantially spherical metabolizing particle. In one embodiment the device is a conventional 48- or 96-well device for cell culturing. However, the conventional wells have openings too large for allow a gradient be established when measuring a substantially spherical metabolizing particle. Therefore, the wells may be provided with insert(s) as described above. The inserts may be positioned during the whole culture period, or only in the period of establishing the metabolite gradient and measuring the metabolite concentration.

### Culturing Device

The present invention further relates to an optimized device for culturing of metabolizing particles as defined above, wherein said device comprises at least one compartment as described above. Accordingly, the invention relates to a device for culture of a metabolizing particle, which device comprises at least one compartment, said compartment being defined by a diffusion barrier and capable of comprising a medium with a metabolizing particle, said diffusion barrier allowing metabolite transport to and/or from the metabolizing particle by means of diffusion, whereby a metabolite diffusion gradient is allowed to be established from the metabolizing particle and throughout the medium.

The compartment is preferably as described above, except that a detector is not necessarily included into the culture device. Accordingly, the device may comprise more than one compartment, such as at least two compartments, such as at least 4 compartments, such as at least 6 compartments, such as at least 8 compartments, such as at least 12 compartments, such as at least 24 compartments, such as at least 48 compartments, such as at least 96 compartments.

The device offers optimized conditions for culturing of cells and organisms in that the microenvironment surrounding the cells and organisms is easily monitored and optimized as described herein.

Accordingly, the invention further relates to a method for culturing a metabolizing particle, said method comprising

a) providing at least one device as defined herein,
b) arranging a metabolizing particle in the medium of the compartment, and
c) culturing the metabolizing particle.

It is within the scope of the invention to combine the culture method with any of the other methods as described herein.

### Method of Determining the Metabolizing Rate

In another aspect the invention relates to a non-invasive method for determining the metabolic rate of a substantially spherical metabolizing particle. Said method comprises
a) providing at least one device as defined above
b) arranging a substantially spherical metabolizing particle in the medium of a compartment,
c) measuring a metabolite concentration inside the compartment obtaining a metabolite concentration measure, and
d) correlating said metabolite concentration measure to a metabolic rate of said substantially spherical metabolizing particle, thereby determining the metabolic rate of the substantially spherical metabolizing particle

The metabolite may be as described above. The metabolite may be supplied to or removed from the substantially spherical metabolizing particle by diffusion through the medium, such as oxygen supplied to the substantially spherical metabolizing particle by diffusion through the medium. In the latter case, the metabolic concentration may be the gas partial pressure, such as the gas partial pressure of oxygen or carbon dioxide.

It is preferred that the substantially spherical metabolizing particle is cultured in the compartment, so that no unnecessary disturbances of the substantially spherical metabolizing particle take place due to the determination of the metabolic rate.

The metabolite concentration may be measured in a volume smaller than the volume of the compartment and/or the volume of the medium. It is preferred that the metabolisation rate of said substantially spherical metabolizing particle is determined by determining a metabolite diffusion gradient in the compartment based on the measured metabolite concentration, and correlating said metabolite diffusion gradient to the metabolic rate of said substantially spherical metabolizing particle.

The metabolic rate may be determined by performing one measurement of the metabolite concentration, or several measurement, such as at least two measurements. Furthermore, the metabolic rate may be determined more than once during the culture period to monitor the status of the substantially spherical metabolizing particle.

As described above, when the metabolite is a gas, such as oxygen, the gas may be supplied to the substantially spherical metabolizing particle by diffusion through the stagnant medium in the compartment directly from the atmosphere or from a larger volume of medium in equilibrium with the atmosphere.

### Closed Respirometry

The device according to the present invention may further be used for measuring the respiration rate of a particle, such as a substantially spherical metabolizing particle by closed respirometry. Closed respirometry is a measure of the respiration rate in a closed respirometric cell, i.e. a cell wherein the supply of oxygen is terminated at least temporarily. The present device can be converted into a closed respirometric cell by applying a cover of a material impermeable to the metabolite, such as oxygen, over any opening(s) in the compartment(s) of the device. The cover may be produced from any of the impermeable materials mentioned above.

Accordingly, the present invention further relates to a non-invasive method for determining the metabolic rate of a metabolizing particle, comprising
a) providing at least one device as defined above,
b) culturing a metabolizing particle in the medium of a compartment,
c) reducing metabolite supply to the medium during at least a part of the culturing period,
d) measuring a metabolite concentration inside the compartment obtaining a metabolite concentration measure after the metabolite supply has been reduced, and
e) correlating said metabolite concentration measure to a metabolic rate of said substantially spherical metabolizing particle.

With respect to closed respirometry, the metabolite is most often oxygen and the metabolic rate is the respiration rate. In the method the oxygen supply is preferably reduced to zero.

It is preferred that the metabolite concentration measure has been obtained during the period of reduced supply.

### Regulation of Metabolite Supply

In a further aspect the invention relates to a method for regulation of metabolite supply to a particle, such as a substantially spherical metabolizing particle, in a compartment.

According, the invention further relates to a method for regulating metabolite supply to a substantially spherical metabolizing particle during culturing, comprising
a) providing at least one device comprising a compartment with medium,
a) culturing a substantially spherical metabolizing particle in the medium of a compartment,
b) measuring a metabolite concentration inside the compartment obtaining a metabolite concentration measure, and optionally
c) correlating said metabolite concentration measure to a metabolic rate of said substantially spherical metabolizing particle.
d) regulating the metabolite supply depending on the metabolite concentration measure and/or the metabolic rate of said substantially spherical metabolizing particle.

The method in particular relates to the measurement, wherein the metabolite is a gas, such as oxygen and the metabolic process is respiration.

The compartment is preferably a compartment as defined herein suitable for allowing establishment of a metabolite diffusion gradient.

The regulation of the metabolite supply may be conducted in any suitable manner. In one embodiment the regulation is conducted by changing the metabolite concentration outside the compartment.

In another embodiment the regulation is conducted by changing the dimensions of the compartment. As described above, the volume may be changed in several ways. One example hereof is wherein the dimensions are adjusted by inserting an insert, such as wherein the transverse dimensions of the compartment is adjusted by inserting an insert. The longitudinal dimension may also be adjusted by shifting the position of an adjustable bottom of the compartment.

In a third embodiment the regulation is conducted by changing the diffusion barrier of the compartment. This may be conducted by changing the thickness of a compartment wall, or by changing the size of at least one opening in the compartment wall.

### Monitoring of Substantially Spherical Metabolizing Particles

The present invention further relates to monitoring of substantially spherical metabolizing particles and selection of substantially spherical metabolizing particles having a high quality in terms of viability as measured by metabolizing rate.

In a preferred embodiment the invention relates to selection of viable embryos, such as a method for selecting a viable embryo comprising,

a) determining the metabolic rate of the embryo at least once during culturing,

b) selecting the embryo having an optimal metabolic rate.

The determination of the metabolic rate is preferably conducted in a device as defined by the present invention as well as by a method as described herein. Furthermore, the embryo is preferably cultured in the compartment of said device.

Thereby it is facilitated that the determination of the metabolic rate is conducted without causing any change in the growth conditions experienced by the embryo.

### DETAILED DESCRIPTION OF THE DRAWINGS

In the following a number of different embodiments of the present invention will be described with reference to the accompanying drawings, but it is to be understood that these embodiments only constitute examples of the general inventive idea, and that other embodiments may be conceivable by a person skilled in the art.

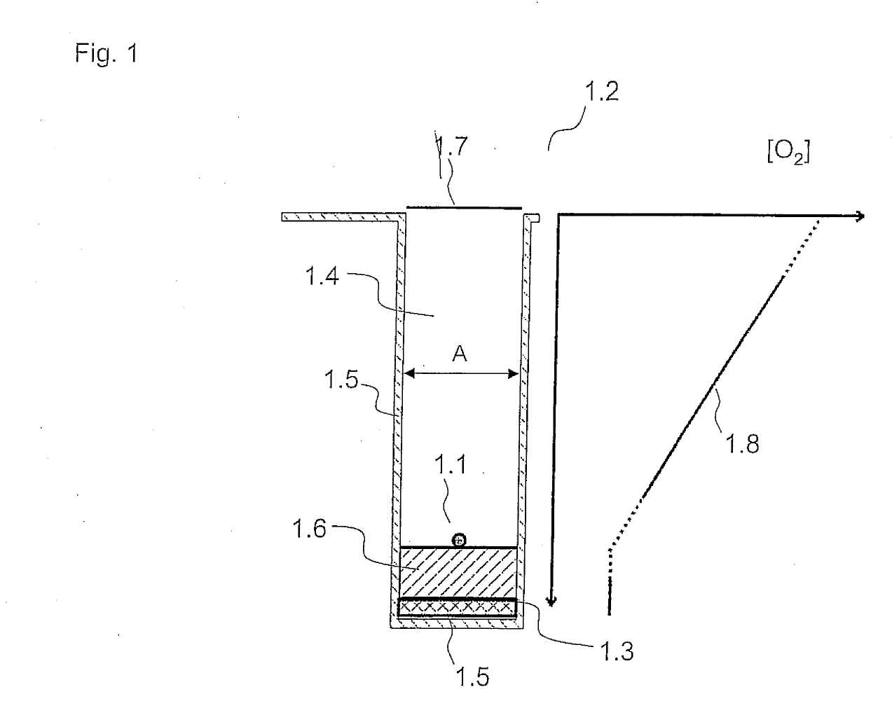

The embodiment of the invention shown in FIG. 1 for measuring embryo respiration illustrates a longitudinal compartment 1.4 open in one end. The bottom of the compartment, which could be cylindrical, consists of a gas permeable substance 1.6 on top of a transparent oxygen sensitive luminophore 1.3. The bottom wall 1.5 of the diffusion compartment is made of a transparent material which allows visual inspection of the embryo under magnification. The bottom wall 1.5 is made of a gas impermeable material like glass or plastic, such that the only supply of oxygen is through the opening of the compartment 1.7. Oxygen partial pressure, in the luminophore layer 1.3 at the bottom of the compartment, is measured by means of an external luminescence reader by recording luminescence from the oxygen luminophore 1.3 at the bottom of the compartment, through the transparent bottom wall 1.5. The surroundings 1.2 which in one embodiment could be bulk medium is in equilibrium with an atmosphere of known or unknown gaseous composition. The device accommodates a single or several embryos 1.1 placed on the gas permeable substance 1.6, which substance in one embodiment is silicone, on top of a transparent oxygen sensitive luminophore 1.3. The gas permeable substance 1.6 can be a silicone compound, a Teflon fluoropolymere, a plastic compound like polyethylene, polypropylene or neoprene, a permeable matrix or porous material based on another chemically inert material like glass, ceramics or minerals, glass or mineral fibers or a precious metal like gold or platinum.

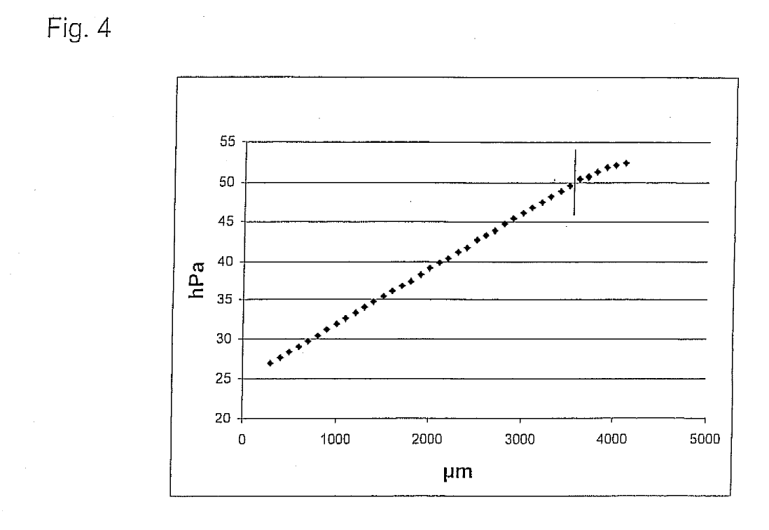

The functional principle of the invention is that the embryo's consumption of oxygen reduces the oxygen partial pressure at the oxygen detector (luminophore) 1.3 compared to the oxygen partial pressure in the bulk medium/surroundings 1.2. The oxygen partial pressure gradient 1.8 will in steady state be stable and not subject to change as long as the embryo's oxygen consumption is constant. In the present embodiment comprising a longitudinal cylindrical diffusion compartment, the oxygen partial pressure gradient will be linear as indicated in FIG. 1. Real experimental data are shown in FIG. 4. The oxygen consumption by the embryo will therefore be determined as the difference between the oxygen partial pressure at the bottom 1.3 and the opening 1.7 of the said diffusion compartment 1.4 using Picks 1. law of diffusion (equation I),

$$J = - D \frac{d C}{d x} \qquad ( I )$$

assuming a linear decrease (see the theoretical graph 1.8) of oxygen from the opening towards the embryo 1.1 at the bottom, where J is the flux of oxygen, which in steady state equals the consumption of the embryo, D is a known diffusion coefficient of the medium and dC/dX is the oxygen gradient. The gradient dC/dX is the difference in oxygen partial pressure between the defined atmosphere or medium at the opening 1.7 of the compartment and the bottom of the compartment at the level of the embryo 1.1. The use in the present embodiment of an optical oxygen luminophore 1.3, covering the bottom of the compartment 1.4, will integrate the oxygen signal over the total bottom area. Horizontal oxygen gradients at the level of the embryo, arising from an unevenly distributed oxygen consumption related to the embryo, will be averaged, as if the consumption was evenly distributed over the bottom area, such that the exact placement of the embryo becomes irrelevant for the respiration estimation.

The respiration rate of an individual embryo can thus be determined by a single oxygen partial pressure measurement, performed from the outside of the diffusion compartment without perturbing the embryo, by oxygen detector means immobilized inside the compartment. The measurement can be performed within a few seconds without any disturbance of the embryo. Depending on the detector means, the measurement can be performed inside the incubator, which could e.g. be an incubator or a warm room, or the measurement can be performed within a very short time outside the incubator, such that growth conditions experienced by the embryo is not significantly affected.

By using an array of compartments of the present invention, multiple embryos can be scanned for individual respiration rates simultaneously. In one embodiment the at least one compartment comprises at least 5 compartments, particularly at least 10 compartments, more particularly at least 24 compartments, and even more particularly at least 96 compartments.

In a further embodiment individual embryos are grown in separate compartments and in a still further embodiment each compartment comprises more than one embryo.

FIG. 2 shows an insert 10 inside the first embodiment, which serves to adjust the transverse dimension A of the longitudinal compartment 2.4. By narrowing or enlarging the transverse dimension the capacity of the diffusion compartment to transport dissolved substances by diffusion can be increased or reduced. The transport capacity of the diffusion compartment determines the steady state oxygen partial pressure at the position of the embryo.

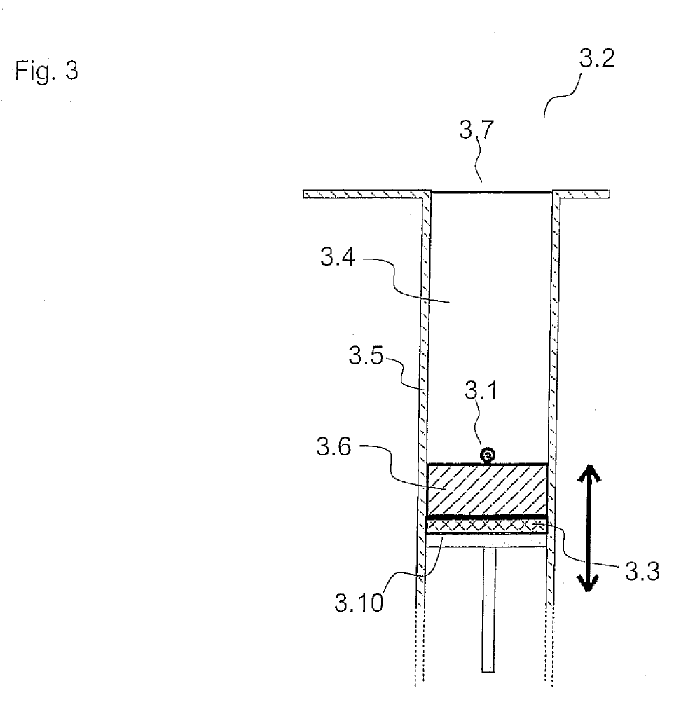

In one embodiment of the invention the oxygen partial pressure at the position of the embryo is controlled by adjusting the dimensions of the compartment 2.4. This can be done in several ways, e.g. by adjusting the position of a adjustable bottom 3.10 (see FIG. 3), by decreasing or increasing the medium level inside the compartment, or by introducing an insert 2.9 (see FIG. 2) into the compartment, which will reduce the transverse dimension A.

The thickness of the gas permeable layer 2.6 is in one embodiment at least 100 μm particularly at least 300 μm, and more particularly at least 900 μm. The thickness of the gas permeable layer should preferably be about twice or more the diameter of the embryo, which for mammalian embryos typically is between 30-400 μm dependent on the developmental stage and species.

FIG. 3 shows another embodiment of the present invention. Elements identical with elements of the first embodiment shown in FIG. 1 are designated by the same reference numbers as on FIG. 1 (see figure legend). The embodiment consists of a compartment 3.4, e.g. a cylindrical compartment, with a opening 3.7 in one end, but with a moveable or adjustable bottom 3.10 with a gas permeable layer 3.6 on top of an oxygen sensitive luminophore 3.3. The bottom wall 3.10 is sealed against the compartment wall 3.5 such that the seal is gas impermeable.

The dimension of the compartment in the second embodiment of the present invention can due to the moveable bottom 3.10 be altered in a controlled way either increasing or decreasing the diffusion length of oxygen from the opening of the compartment 3.7 to the embryo 3.1. By increasing or decreasing the length of the compartment 3.4, the steady state oxygen partial pressure at the level of the embryo 3.1 can be either decreased or increased to reach a desired oxygen partial pressure, without affecting the possibility of performing a respiration estimation. The respiration rate of the embryo can in this way be determined with the option of adjusting the oxygen partial pressure as experienced by the embryo to a desired level, at any respiration rate.

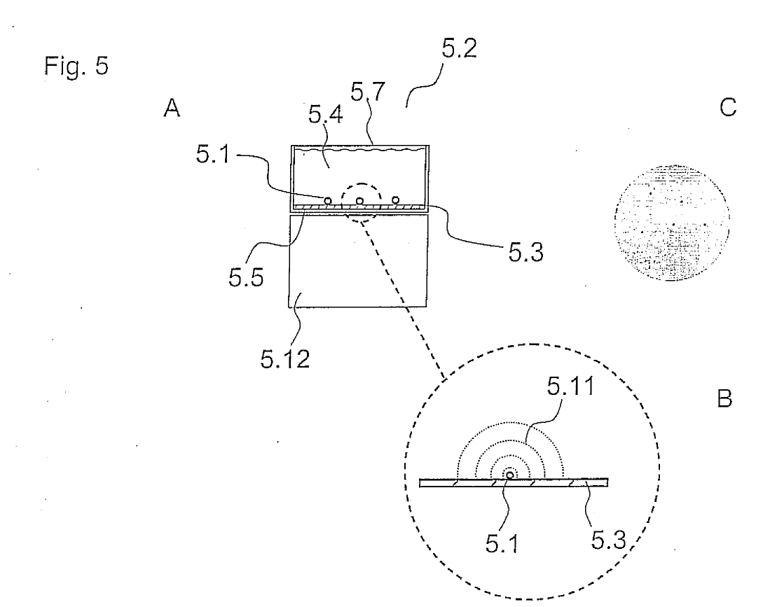

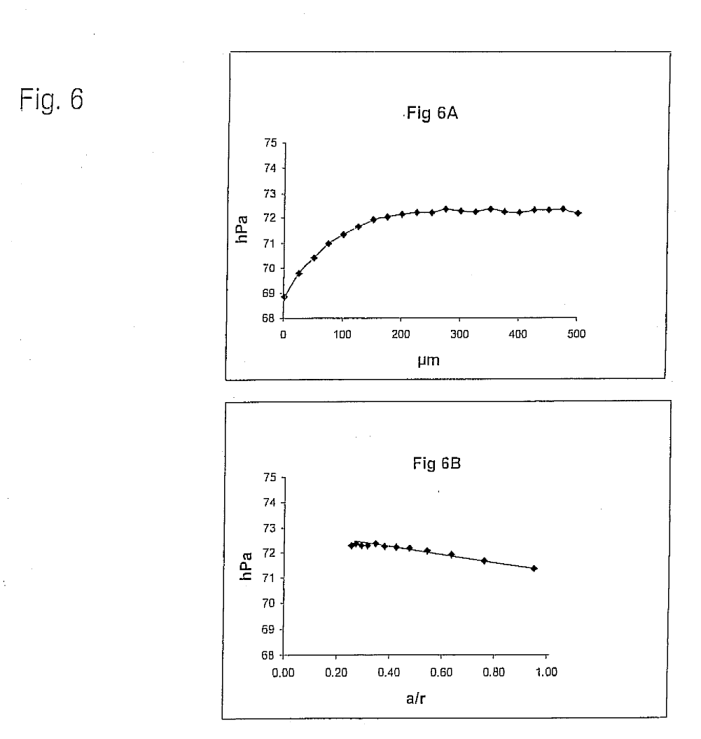

FIG. 5 is yet another embodiment of the present invention where the complete volume of the incubation medium within a growth dish defines the compartment 5.4, which is then much larger than in the other embodiments of the present invention. The bottom 5.5 of the growth dish is transparent and covered with a luminophore 5.3 on top of which is placed one or several embryos 5.1 at a distance from each other, large enough, typically more than 2 mm, to avoid overlap of partial pressure gradients among the embryos. The functional principle of the present embodiment is that oxygen is supplied to the embryo from the surrounding medium in contact with the atmosphere outside the compartment above the embryo as illustrated in FIG. 5 B. When the compartment is very large relative to the embryo, the resulting oxygen gradient towards the embryo will be spherical as illustrated by the oxygen partial pressure iso-lines 5.11 in FIG. 5B, and real data from FIG. 6 The growth dish constituting the diffusion compartment is placed on a CCD camera 5.12 which by optical oxygen sensing resolves the horizontal distribution of oxygen in the luminophore 5.3 in two dimensions. The signal from the CCD camera 5.12 corresponding to the area around each embryo 5.1 will thus become a measure of the individual embryo respiration. The effect is shown in FIG. 5C, which shows an image as seen from the CCD camera, where the luminescence intensity of the luminophore around each individual embryo is visualized in grey tones. Embryo respiration is estimated by fitting a recorded oxygen partial pressure gradient around the embryo to a theoretical model assuming ideal spherical diffusion. The gradient of oxygen towards an oxygen consuming body in a free diffusion space can be described theoretically: The concentration C at a given point r in a hollow sphere between a and b (a<r<b) can be described if the concentration at a (C1) and at b (C2) is know (Crank 1997). There is no consumption of oxygen between a and b.

$$C ( r ) = \frac{{aC}_{1} ( b - r ) + {bC}_{2} ( r - a )}{r ( b - a )} \qquad ( equation II )$$

The flux of oxygen diffusing through the spherical wall J is given by

$$J = 4 π D \frac{ab}{b - a} ( {C}_{2} - {C}_{1} ) \qquad ( equation III )$$

Where D is the diffusion coefficient of oxygen in the media.

The gradient is symmetrical around the oxygen consuming body and can be mirrored at any plane through the center of the body. It is hence possible to consider an oxygen consuming body, in this case an embryo, placed on a plane surface at the bottom of a large compartment (Diameter>1 cm and height more than 2 mm), as the center of a sphere, only such that the oxygen consumed by the embryo will be supplied from a half sphere. The calculated respiration rate (flux of oxygen through the spherical wall), when fitting the recorded gradient to the theoretical model, should therefore be divided by two. If the nature of the gradient in the diffusion compartment, caused by the embryo respiration, can not be described fully, the device can be calibrated by using artificial embryos with a known oxygen consumption. The embodiment is also suitable for a relative comparison of respiration rates among embryos cultured on the same compartment bottom with a 2D recording of oxygen distribution.

### EXAMPLES

### Example 1

A bovine embryo was placed at the bottom of a cylindrical compartment with a diameter of 1 mm and a depth of 4 mm and cultured under an atmosphere with an oxygen partial pressure of 55 hPa. The steady state oxygen partial pressure gradient inside the compartment was measured with 100 μm intervals from the opening of the compartment towards the embryo. The time (in seconds) before steady state is achieved can be approximated by the following formula,

$$t = \frac{0.45 {l}^{2}}{D} ( From J . Crank 1995 , The Mathematics of Diffusion )$$

where l is the depth of the diffusion compartment in cm and D is the diffusion coefficient of the medium. Steady state in a compartment with a diameter of 1 mm and a depth of 4 mm will thus be achieved after approximately 35 minutes assuming a D of 3.5×10⁻⁵. A Clarck type oxygen micro sensor with a tip size of 10 μm, positioned with a micromanipulator, was used. The data, as shown in FIG. 4, show a linear gradient through the compartment. It is thus sufficient to know the oxygen partial pressure at the top and the bottom of the compartment to determine the gradient. From FIG. 4 it is furthermore obvious that the gradient can be determined by measuring the oxygen partial pressure at any point along the linear gradient inside the compartment, from the opening towards the embryo.

For practical purposes, and when using a luminophore layer, it is more convenient to place the means for oxygen detection in the bottom of the compartment.

### Example 2

After the embryo manipulation, each individual embryo is transferred by pipette to a compartment (In vitro fertilization, cloning, thawing or another technique. See e.g.: In vitro fertilization. Kay Elder, Brian Dale, 2nd rep. Ed, Cambridge University Press (2001), for a general description of embryo manipulation techniques). The compartment is comprised within a larger frame with several compartments, such that one or several batches of embryos, from one or several humans or animals, can be contained in a single frame with multiple compartments, or groups of compartments. The frame is then incubated under desired conditions, which for human embryos typically would be 37° C., 5-21% O₂ and 5% CO₂ in N₂, 100% humidity, grown in commercially available culture medium (e.g. IVF-50 from Scandinavian IVF Science AB, Göteborg, Sweden). The medium of choice depends on the acceptance of quality control and availability of media rather than any specific type. Relatively simple balanced salt solutions for culture of embryos can be used. Earle's, Tyrode's and Hepes media have been successfully introduced. These media are available commercially as single strength or concentrated solution. The respiration measurement is performed by placing the frame in a specially designed luminescence reader, which yields a luminescence signal from the luminophore at the bottom of each individual compartment. The frame is returned to the incubator immediately after the measurements. The actual respiration rate is calculated with information about each individual compartment dimension. If the oxygen partial pressure at the position of the embryo is not within a given optimal interval, e.g. between 5-10%, the compartment dimensions, and thus oxygen partial pressure, is adjusted e.g. with an appropriate insert.

The respiration measurement is performed as often as required during the in vitro culture period. The embryos respiration rate, typically in combination with a morphological evaluation, is then used as the basis for selection of the embryos for transfer to the recipient.

Morphological evaluation in vitro is based on several features of the embryo. Such evaluation methods are subjective and depend very much on experience. The embryo is spherical and is composed of cells (blastomeres) surrounded by a gelatine-like shell, an acellular matrix known as the zona pellucida. The zona pellucida performs a variety of functions until the embryo hatches, and is a good landmark for embryo evaluation. The zona is spherical and translucent, and should be clearly distinguishable from cellular debris. The important criteria in a morphological evaluation of embryos are: (1) shape of the embryo; (2) presence of a zona pellucida; (3) size; (4) colour; (5) knowledge of the age of the embryo in relation to its developmental stage, and (6) blastomere membrane integrity. During embryonic development, blastomere numbers increase geometrically (1-2-4-8-16- etc.). Synchronous cell division is generally maintained to the 16-cell stage in embryos. After that, cell division becomes asynchronous and finally individual cells possess their own cell cycle. The cells composing the embryo should be easily identified by the 16-cell stages as spherical cells. After the 32-cell stage (morula stage), embryos undergo compaction. As a result, individual cells in the embryo are difficult to evaluate beyond this stage. Human embryos produced during infertility treatment are usually transferred to the recipient before the morula stage, whereas other mammalian embryos often are cultured experimentally to a further development stage (expanded blastocysts) before transfer to the recipient or discharge.

### Example 3

Modeled semi-spherical diffusion: FIG. 6A shows an oxygen profile towards a bovine embryo lying on the flat bottom of a large compartment. FIG. 6B displays the same data in C(r) versus a/r, where a is the distance from the sphere center (center of embryo) to the chosen endpoint (towards the embryo) of the oxygen profile. In case the profile starts at the surface of the embryo, a is the radius of the embryo (a can be chosen also at a point distant from the embryo). The assumption about spherical diffusion is fulfilled if the C(r) versus air is linear, for very large b values (when C2 is the true bulk concentration).

The flux of oxygen passing through the sphere at point a can be calculated as described previously. FIG. 6B shows that the assumption of perfect spherical diffusion is not completely fulfilled in this particular case, as the line is not completely linear. The consumption estimate will hence be influenced by the choice of a, which is not the case for a perfect fit.

### Example 4

### Diffusion Theory

### 1. Diffusion Towards a Constant Source of Consumption.

In a diffusion system with a metabolizing particle or object that consumes a compound, the compound will be transported towards the consumption source by diffusion. The magnitude of this diffusion—the flux—can be described by Fick's First Law:

$$J = - D \frac{d C}{d x} , ( 4.1 .1 ) ( Crank , 1997 )$$

where D is the diffusion coefficient, C is the concentration, and x is the axis along which the flux is considered.

At steady state, the area-integrated diffusional flux towards the consumption source at any position in the system will be constant. The area integrated flux is defined as the cross-sectional area, F, of the diffusion system perpendicular to the axis of symmetry. Given the positive consumption, Q this can be expressed as:

−J·F=Q,

which by substitution with equation 4.1.1 yields

$$D \frac{d C}{d x} \cdot F = Q \qquad ( 4.1 .2 )$$

Below these equations will be applied to a range of geometries (parallel-sided polyhedra, cylindrical, spherical), that can be considered to represent different embodiments of the present invention. For example calculations, the oxygen respiring particle is suspended in water with a diffusion coefficient of 3.45·10⁻⁵ cm² s⁻¹ (at 38° C.)

### 2. One-Dimensional System (Parallel-Sided Polyhedra or Cylinder)

A diffusion system is defined as one-dimensional, if the concentration of the diffusing compound and the physical boundaries only vary in one dimension. An infinitely wide plane sheet is an example of a one-dimensional system. If edge effects can be ignored, a parallel-sided well with a source of consumption at the bottom is to be considered as one-dimensional diffusion system.

At steady state, one-dimensional diffusion can be described mathematically as:

$$\frac{{d}^{2} C}{d {x}^{2}} = 0 ( Crank , 1997 ) ,$$

which upon integration yields

$$\frac{d C}{d x} = A , \qquad ( 4.2 .1 )$$

which upon a further integration yields

C=Ax+B (4.2.2)

where A and B are integration constants.

Consider a one-dimensional system with a length h which has a constant concentration C_{w} at x=h, and a source of constant consumption Q at x=0. In a one-dimensional diffusion system, the cross-sectional area, F, is constant as a function of x.

Applying equation 4.2.1 on equation 4.1.2 yields

D·A·F=Q (4.2.3)

The concentration at x=h equals C_{w}, which according to eq. 4.2.2 yields:

C(h)=Ah+B=C_{w} (4.2.4)

By combining equations (4.2.3) and (4.2.4) to solve for A and B, equation (4.2.2) can be rewritten:

$$C ( x ) = {C}_{w} + \frac{Q}{F \cdot D} ( x - h ) , \qquad ( 4.2 .5 )$$

Considering the concentration C₀ at x=0, equation 4.2.5 can be rearranged to yield:

$$Q = \frac{( {C}_{w} - {C}_{0} ) F \cdot D}{h} . \qquad ( 4.2 .6 )$$

Thus, if F, D, h, and C_{w} are known, the consumption rate can be calculated from a measurement of C₀ using eq. 4.2.6.

Applying this to the measurements performed in Example 6, where the diffusion system was in the form of a parallel-sided cylindrical well with a diameter of 0.5 millimeter, corresponding to yielding a surface area F of 0.00196 cm², and a depth h of 4 millimeter, the measured concentration of 17% oxygen (corresponding to 169 μM) at the bottom of the well compared to 21% oxygen (corresponding to 210 μM) at the top of the well can be translated to a consumption rate of 6.77·10⁻⁶ nmol·s⁻¹ corresponding to 0.546 nanoliters·hour⁻¹.

### 3. Cylindrical System (Disk-Shaped) Essentially Two Dimensional.

In a cylindrical diffusion system, diffusion takes place along the radius of the cylinder, whereas there is no change along the longitudinal axis of the cylindrical system. If edge effects can be neglected, a diffusion system consisting of a disk-shaped body with a consumption source in its center is to be considered a cylindrical system.

At steady state, cylindrical diffusion can be described mathematically as:

$$\frac{d}{d r} ( r \frac{d C}{d r} ) = 0 ( Crank , 1997 ) ,$$

which upon integration yields

$$r \frac{d C}{d r} = A , \qquad ( 4.3 .1 )$$

which upon a further integration yields

C=A+B1nr (4.3.2)

where A and B are integration constants.

Consider a cylindrical system with a length l and radius r₁ which has a constant concentration C_{w} at r=r₁, and a source of constant consumption Q at r=0.

Applying equation 4.3.1 on equation 4.1.2 yields

$$D \cdot \frac{A}{r} \cdot F = Q \qquad ( 4.3 .3 )$$

The concentration at r=r₁ equals which according to eq. 4.3.2 yields:

C(r₁)=A+B1nr₁=C_{w} (4.3.4)

In a cylindrical diffusion system, the cross-sectional area, F, is a function of r:

F=2π·r·l (4.3.5)

By combining equations 4.3.3, 4.3.4, and 4.3.5 to solve for A and B, equation (4.3.2) can be rewritten:

$$C ( r ) = {C}_{w} + \frac{Q}{2 π \cdot l \cdot D} ln ( r / {r}_{1} ) , \qquad ( 4.3 .6 )$$

Considering the concentration C₀ at an additional point, r_{O}, in the diffusion system, equation 4.3.6 can be rearranged to yield:

$$Q = \frac{( {C}_{w} - {C}_{0} ) \cdot 2 π \cdot l \cdot D}{ln ( {r}_{1} / {r}_{0} )} . \qquad ( 4.3 .7 )$$

Thus, if l, D, r₀, r₁, and C_{w} are known, the consumption rate can be calculated from a measurement of C₀ using eq. 4.3.7.

If a cylindrical diffusion system is constructed by placing a 10 mm diameter circular impermeable disk 50 μm above an impermeable surface and placing an oxygen respiring particle with a diameter of 100 μm and an oxygen respiration rate of 1 nl oxygen hour⁻¹ under the center of the disk, the resulting steady-state concentration will be 157 μM at the particle surface according to Eqn. 4.3.6.

### 4. Spherical System (Cone-Shaped—Hemispherical A Three Dimensional System.

In a spherical diffusion system, diffusion takes place along the radius of a sphere or a section of a sphere. If edge effects can be neglected, a diffusion system consisting of a cone-shaped body with a consumption source a its tip is to be considered a spherical system.

At steady state, spherical diffusion can be described mathematically as:

$$\frac{d}{d r} ( {r}^{2} \frac{d C}{d r} ) = 0 ( Crank , 1997 ) ,$$

which upon integration yields

$${r}^{2} \frac{d C}{d r} = A , \qquad ( 4.4 .1 )$$

which upon a further integration yields

$$C = B + \frac{A}{r} , \qquad ( 4.4 .2 )$$

where A and B are integration constants.

Consider a spherical system with a radius r₁ which has a constant concentration C_{w} at r=r₁, and a source of constant consumption Q at r=0.

Applying equation 4.4.1 on equation 1.2 yields

$$D \cdot \frac{A}{{r}^{2}} \cdot F = Q \qquad ( 4.4 .3 )$$

The concentration at r=r₁ equals C_{w}, which according to eq. 4.4.2 yields:

$$C ( {r}_{1} ) = B + \frac{A}{{r}_{1}} = {C}_{w} . \qquad ( 4.4 .4 )$$

In a cylindrical diffusion system, the cross-sectional area, F, is a function of the radius, r. If the system consists of a cone, F can be described as:

$$F = 2 π \cdot {r}^{2} \cdot ( 1 - cos \frac{θ}{2} ) , \qquad ( 4.4 .5 )$$

where θ is the tip angle of the cone. By combining equations 4.4.3, 4.4.4, and 4.4.5 to solve for A and B, equation (4.4.2) can be rewritten:

$$C ( r ) = {C}_{w} - \frac{Q}{2 π \cdot D \cdot ( 1 - cos \frac{θ}{2} )} ( \frac{1}{r} - \frac{1}{{r}_{1}} ) , \qquad ( 4.4 .6 )$$

Considering the concentration C₀ at an additional point, r₀, in the diffusion system, equation x4.6 can be rearranged to yield:

$$Q = ( {C}_{w} - {C}_{0} ) \cdot 2 π \cdot D \cdot ( 1 - cos \frac{θ}{2} ) {( \frac{1}{{r}_{0}} - \frac{1}{{r}_{1}} )}^{- 1} . \qquad ( 4.4 .7 )$$

Thus, if D, r₀, r₁, θ, and C_{w} are known, the consumption rate can be calculated from a measurement of C₀ using eq. 4.4.7.

Applying this to the measurements performed in Example 7, where a C₀=206 μM was found at r_{0,}=0.015 cm (embryo surface), and where D=3.45·10⁻⁵ cm² s⁻¹, r₁=0.04 cm, θ=60°, and C_{w}=210 μM, yields a respiration rate of 0.11 nl hour⁻¹.
Literature: Crank, J. 1997. The Mathematics of Diffusion. Clarendon Press.

### Example 5

### Design Examples of Novel Devices Described by the Invention

The figures in this patent application show 15 different designs for the novel devices described here. Many of these variations are functionally equivalent designs to facilitate handling of the metabolising particle and/or adjusting the diffusion barrier to ensure optimal incubation conditions for the metabolising particle. They can be organized into categories according to the metabolite concentration gradient type that is generated in the media within and in close proximity to the compartment. The four categories are:
1. One dimensional system with a linear metabolite concentration gradient
2. Two dimensional system with a logarithmic metabolite concentration gradient
3. Three dimensional system (cone—hemisphere) with a hyperbolic concentration gradient
4. Irregular systems which is a combination of the above, and where more complex modelling is required to describe the concentration gradient

The diffusion theory pertaining to each of the three categories is described in detail in the previous example (Example 4). Using the derived equations, we present an example of how to design and dimension an example from each category of devices to obtain a desired sensor signal for a metabolising particle with a given respiration rate. Our standard example deals with oxygen diffusion to a respiring embryo suspended in aqueous medium. We will use the following standard parameters:
consumption rate, Q=1.0 nanoliters·hour⁻¹ (=to 1.24·10⁻⁵ nmol·s⁻¹),
oxygen diffusion coefficient. D=3.45·10⁻⁵ cm² s⁻¹ in medium (at 38° C.).
an oxygen concentration in the medium, C_{w},=210 μM at 38° C.
desired sensor signal is 30% lower than bulk (i.e. C₀=147 μM at 38° C.)
   One Dimensional System with a Linear Metabolite Concentration Gradient

The diffusion equations pertaining to such a system is described in the second section of Example 4. If we assume a height of the cylinder, h, of 3 mm then we can use Eqn. 4.2.6 to calculate the diameter, d, of the cylinder, given that F=π(d/2) [symbol] d=2\sqrt{F/π}

$$d = \sqrt{\frac{4 \cdot Q \cdot h}{π \cdot D \cdot ( {C}_{w} - {C}_{0} )}} . \qquad ( 5.1 )$$

Given the standard parameters above we find the diameter of the cylinder must be reduced to about 470 μm to give the desired signal for an object with the expected oxygen respiration rate.

Design A, shown in FIG. 1 Bore in an impermeable material. This design is a simple cylindrical bore in an impermeable material (1.5). It could also be a rectangular or polyhedral cavity with similar dimensions. The metabolising particle (1.1) is placed at the bottom on a layer of a permeable material 1.6 above a detector (1.3) (which could be, but is not limited to, a layer of luminophore that is observed from the top or through the transparent bottom (1.5)). The purpose of the permeable layer (1.6) is to even out the horizontal metabolite concentration gradient found in close proximity to the metabolising particle (1.1). The observed signal from the detector (1.3) will thus be practically uniform across its surface. Concentration gradients in close proximity to the metabolising particle will deviate slightly from an ideal linear curve, but if the aspect ratio of the bore is high (i.e. h>>d) then these slight distortions are insignificant. Outside the opening (1.7) we expect a hemispherical gradient in which the concentration quickly assumes the concentration of the bulk fluid. For all practical purposes we can thus consider the system as a one dimensional system with properties described by the equation above, where the flux is controlled by the aspect ratio of the bore. The diameter of the bore should be kept so low that turbulent mixing does not occur in the media column above the metabolising particle. The advantage of this design is its simplicity. It has successfully been used for the oxygen microelectrode experiments described in Example 1. The two main disadvantages with this design is: 1) the difficulty to retrieve a metabolising particle deposited in a deep narrow bore, 2) the inability to regulate the diffusion barrier based on the metabolic rate of the metabolising particle

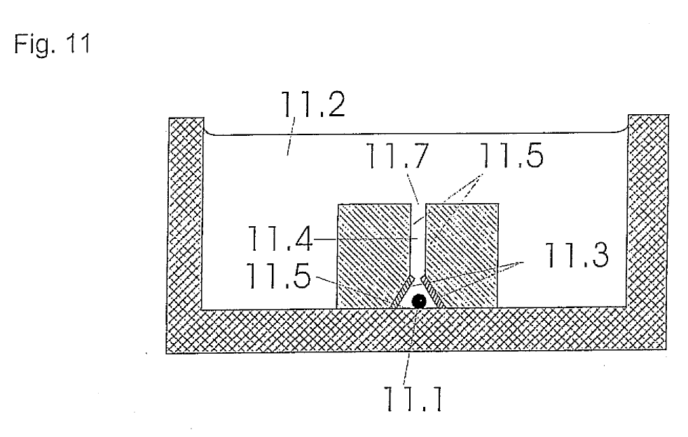

Design B, shown in FIG. 11. Bore with exchangeable top. This design is very similar to the simple bore discussed previously (design A). It is composed of two impermeable pieces. A vessel made of an impermeable material (e.g. glass) filled with medium (11.5). Onto this vessel is placed a small piece of an impermeable material (11.5) with a cylindrical (or polyhedral) hole (11.4) through its centre. At the end of the hole facing the vessel surface the hole is excavated (“hollowed out”) to form a small cavity into which the respiring particle (11.1) is placed. The top walls of the cavity are covered with metabolite detectors (11.3). As long as the cavity “chamber” in which the metabolising particle is placed is small and the aspect ratio of the hole is high, then the design is equivalent to the simple bore discussed above. A prototype of this design made of glass pieces with an oxygen sensitive luminophore has successfully been used to measure respiration rates of murine embryos as described in Example 6. The advantage of this device compared to the central bore described above is the possibility to remove the metabolising particle from the device by separating the two impermeable pieces (i.e. removing the top “chimney”). It is also possible to exchange tops to provide a different diffusion barrier with a different diameter or length of the central bore. However, the main disadvantage is the difficulty to make the two impermeable parts fit smoothly, so the there are no horizontal gaps at the interface between them wherein the metabolite can diffuse horizontally along the interface.

Design C, shown in FIG. 2. Bore with insert. This design is identical to design A (FIG. 1). The only difference is an impermeable insert into the bore (2.9), which reduces the cross section of the bore from A in FIGS. 1 to 13 in FIG. 2. The reduced cross section will increase the diffusion barrier and thus reduce the metabolite concentration in the compartment below the insert (2.4). The main advantage of this design is the ability to regulate the diffusive barrier by changing between inserts with different bore diameters. It may also be easier to remove the metabolising particle if the insert is first removed to increase the diameter of the bore and facilitate access to the metabolising particle. A disadvantage is the required size of inserts and bores that makes them very difficult to handle as they are very small and must fit very well to avoid gaps between insert and bore through which the metabolite could diffuse.

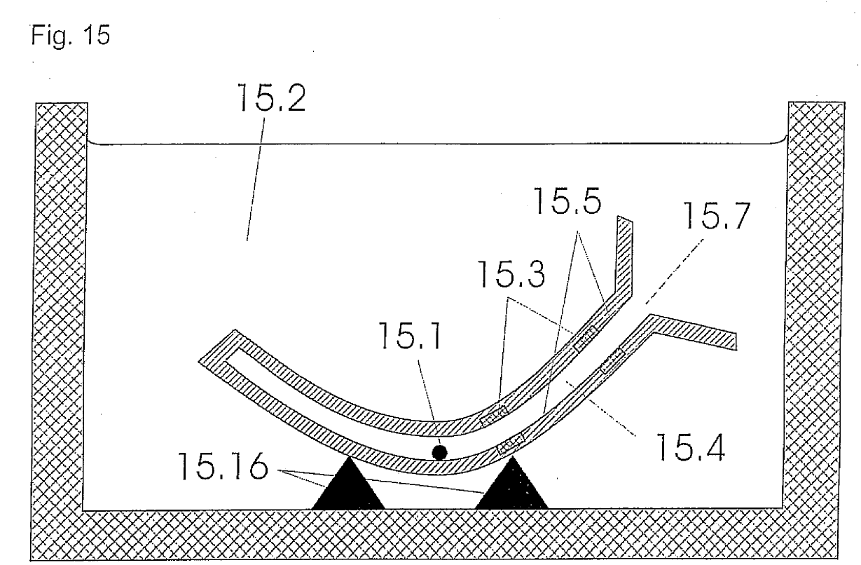

Design D, shown in FIG. 15 Bent capillary. This design is functionally equivalent to design A. It consists of a bent impermeable capillary (15.5) with one closed end (or a very distant opening so that diffusive transport of metabolite from the back end can be neglected) and a funnel at the other end. A metabolising particle is placed at the funnel end (15.7) and allowed to settle by gravity at the lowest point (15.1) of the capillary which is placed on two holders (black triangles marked 15.4) submerged in a vessel containing medium (15.2). A metabolite sensitive detector is placed in two bands (15.3) to detect the metabolite concentration gradient. If there are two bands then the outermost band may serve as a reference and the distance from the opening (15.7) to the metabolising particle (15.1) need not be known as long as the bands are more proximal to the opening than the respiring particle. If there is a band at a larger distance from the opening (15.7) than the metabolising particle (15.1) then the distance between the latter and the former must be known. The diameter of the capillary must be small enough to prevent turbulence. An advantage of this design is the ability to regulate the diffusion barrier by tilting the capillary in the holder so that gravity moves (rolls) the metabolising particle (15.1) towards or away from the opening (15.7) thus changing the distance the metabolite has to traverse by diffusion. The largest problem with this device is to get the metabolising particle into the capillary. The funnel should help but will make such a device more difficult to construct.

Design E, shown in FIG. 3. Bore with adjustable bottom. This design is identical to design A, a cylindrical (or polyhedral) bore (3.4) in an impermeable material (3.5) with the metabolising particle (3.1) resting on the bottom permeable layer (3.6) on top of the metabolite sensitive detector (3.3). However, this design employ a piston (3.10) to obtain an adjustable height, h, of the diffusion barrier it is thus possible to adjust the aspect ratio of the bore and hence the metabolite concentration at the bottom of the bore. The adjustable height is here accomplished by moving the bottom with fixed walls, however an identical effect can be achieved by keeping the bottom stationary and moving the walls downwards (as in design G below). The adjustable bottom (3.10) serves two purposes: 1) regulating metabolite supply to the respiring particle by altering the diffusion barrier, and 2) to facilitate the removal of the metabolising particle from the device. The main disadvantage is the required diminutive diameter of the bore and the resultant small size of the piston. It is a further complication that they must fit very well to avoid gaps between piston and bore through which the metabolite could diffuse. It may also be difficult to measure the signal from the metabolite sensitive detector (3.3) from the bottom i.e. through the piston (3.10).

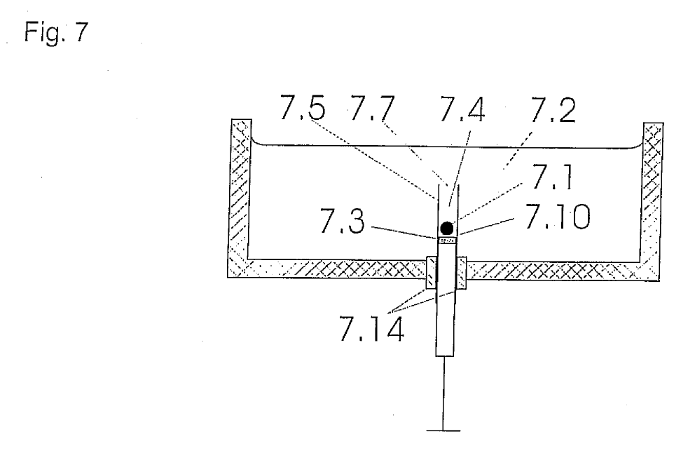

Design F, shown in FIG. 7. Pipette with detector piston. This design is an example of how the design E described above could be realized. It shows a particular embodiment where the adjustable bottom is generated with a pipette, with which the studied metabolising particle is picked up from a transfer container. The plunger of the pipette (7.10) is particular in that it contain a metabolite detector (7.3). After the respiring particle (7.1) has been picked up, the pipette is turned with the tip up and inserted through a port (7.14) in the bottom of a media vessel. The media vessel is subsequently filled with medium (7.2). The barrel of the pipette serves as the sidewalls (7.5) of the compartment.

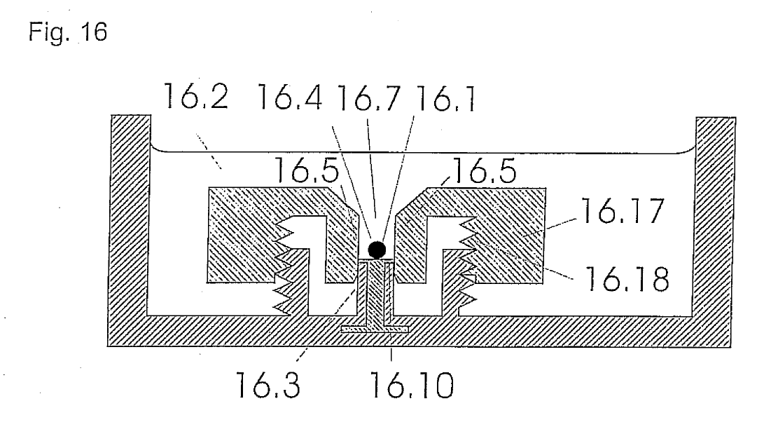

Design G, shown in FIG. 16. Bore with threaded adjustable bottom. This design is functionally equivalent to the two previous, but the adjustment is accomplished in a slightly different way. It contain a central bore (16.4) with adjustable dimensions as the height of the surrounding walls (16.5) can be modified relative to the fixed bottom (16.10) by turning the adjustable top (16.17). The thickness of the diffusion barrier i.e. the thickness of the liquid layer (16.4) is reduced by turning (16.17) clockwise, whereby (16.17), by means of a thread (16.18), is moved towards the bottom of the large well containing surrounding medium (16.2). As the bottom of the compartment (16.10) is fixed relative to the bottom of the larger well this results in a decrease of the compartment volume and thus in a decreased thickness of the permeable layer (16.4) and therefore also in an increased permeability. The detector (16.3) extends from the bottom of the compartment towards the bottom of the larger well containing surrounding medium, where it can be brought in contact with a recording unit. As the detector material (16.3) is embedded in the impermeable well material except for the detector surface (16.10) below the metabolising particle (16.1). The same metabolite concentration should be observed within all of the detector material. It is thus possible to extend the detector into a horizontal disc beneath the metabolising particle. This disc may serve as a physical signal amplifier for an optical detection principle using a metabolite sensitive luminophore embedded in the impermeable material yet observed from below. This type of signal amplification will lead to a slower response of the detector as the whole detector volume act as a reservoir for the metabolite that must be in equilibrium to get a steady state signal. Still this type of passive signal amplification may be useful in other designs as well. The main advantage of the presented design is the gradual adjustment of the diffusion barrier and the easy manipulation of the metabolising particle, when the top is turned all the way down. The main disadvantage is the required diminutive diameter of the bore and the resultant small size of the piston. It is a further complication that they must fit very well to avoid gaps between piston and bore through which the metabolite could diffuse.

Two Dimensional System with a Logarithmic Metabolite Concentration Gradient

In the designs of this category the metabolising particle is positioned between two impermeable planar surfaces so that the permeable material (e.g. media) constitutes a disk. We have essentially radial diffusion in a disc shaped cylinder. In a cylindrical diffusion system, diffusion takes place along the radius of the cylinder, whereas there is no change along the longitudinal axis of the cylindrical system. If edge effects can be neglected, a diffusion system consisting of a disk is developed. The diffusion equations pertaining to such a system is described in the third section of Example 4. If we assume a radius of the detector disk below the metabolising particle to be, r₀=0.5 mm, and the outer radius of the planar surface laying above the metabolising particle to be, r₁=5 mm. Then we can use Eqn. 4.3.7 to calculate the distance, l, between the impermeable planar surfaces to obtain a desired detector signal

$$l = \frac{Q \cdot ln ( {r}_{1} / {r}_{0} )}{2 π \cdot D \cdot ( {C}_{w} - {C}_{0} )} . \qquad ( 5.2 )$$

Given the standard parameters above we find the distance between the planar surfaces must be 20.1 μm to give the desired signal for an object with the expected oxygen respiration rate.

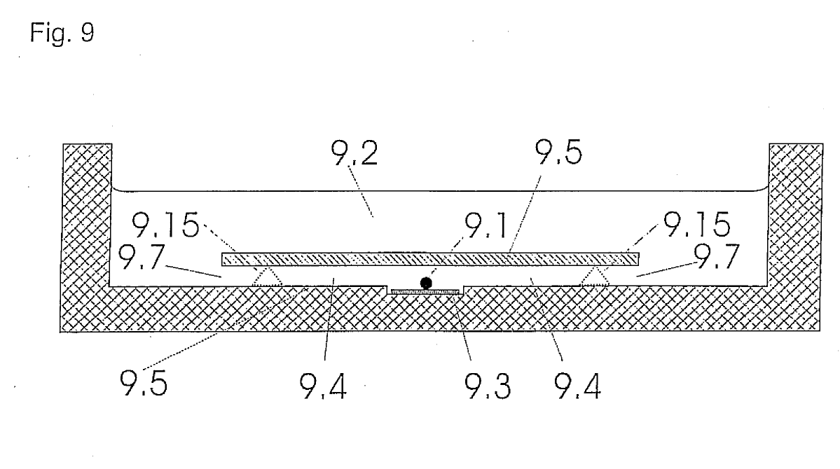

Design H shown in FIG. 9. Diffusion disk between impermeable plates. This is a design where the metabolising particle (9.1) is placed near a detector (9.3) under an impermeable disk (9.5). The disk, which constitutes the upper part of the substantially impermeable compartment wall, is supported by spacers (9.15) to keep a well-defined distance to the lower part of the substantially impermeable compartment wall (9.5). The metabolising particle (p.1) is placed in a shallow well in a plate. The spacers (9.15) are shown with a hatched line to indicate that they only occupy a small fraction of the area under the disk and do not constitute a significant barrier to diffusion. The diffusion barrier can be adjusted by changing the height of the spacers supporting the upper wall (lid) of the substantially impermeable compartment walls. The main disadvantage is the need for highly planar surfaces so that the distance between the plates remains unaltered. Deviation from planarity must only be a few μm over a relatively large diameter of 10 mm, to avoid compromising the uniformity of the gab between the impermeable surfaces. Another problem is placing and removing the tight fitting lid without turbulence disturbing the metabolising particle too much.

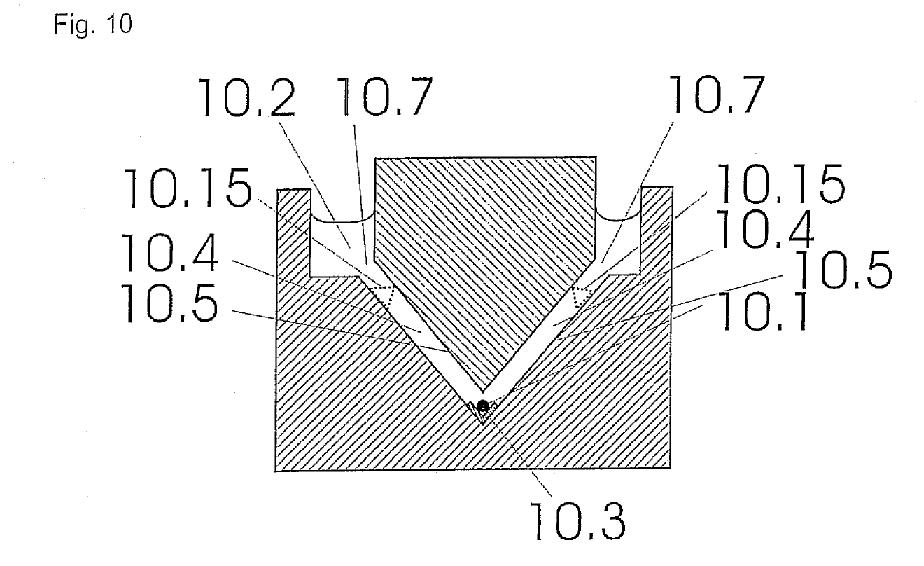

Design I, shown in FIG. 10. Diffusion disk wrapped as the surface of a cone. This is a design, which is functionally equivalent to the previous design as the permeable space available for diffusive transport of the metabolite is confined between two impermeable surfaces. However, in this case the impermeable surfaces are not planar but constitute an impermeable cone lid (10.5) inserted in a cone shaped cavity in an impermeable plate (10.5). The metabolising particle (10.3) is placed in the medium filled (10.2) cone shaped cavity, where it by gravity comes to rest at the bottom tip of the cavity. A detector (10.3) is located near the metabolising particle at the tip of the cavity, and a cone-shaped impermeable lid is placed in the cavity. Spacers (10.15) ensure that a well-defined distance is kept between the lid and the impression. The advantage of this design over the previous is that the metabolising particle density makes it sink to the detector position at the bottom tip of the cavity. Still the spacing between the two impermeable parts must be carefully controlled with very small tolerances, which is not easily accomplished.

Three Dimensional System (Conus—Hemisphere) with a Hyperbolic Concentration Gradient

In the designs of this category the metabolising particle is positioned on a planar impermeable surface or in a conical depression in such a surface. Both cases are examples of a spherical diffusion system, where the impermeable material restricts the angles from which the metabolite can approach the metabolising particle. If we express the observed flow as a function of the angle of the conical depression, then the hemispherical diffusion pattern of a metabolising particle on a impermeable surface can be described by the same set of equations with the angle θ=180°. The diffusion equations pertaining to such a system is described in the fourth section of Example 4. If we assume a radius of the cone at the position of the metabolising particle to be, r₀=0.5 mm, and the outer radius of the conical depression to be, r₁=3 mm. Then we can use Eqn. 4.4.7 to calculate the conical angle, 0, at the tip of the cone to obtain a desired detector signal

$$θ = 2 \cdot {cos}^{- 1} [ 1 - \frac{Q ( \frac{1}{{r}_{0}} - \frac{1}{{r}_{1}} )}{2 π \cdot D \cdot ( {C}_{w} - {C}_{0} )} ] . \qquad ( 5.3 )$$

Given the standard parameters above we find the conical angle must be 20° to give the desired signal for an object with the expected oxygen respiration rate. This corresponds to a 2.5 mm deep conical hole with an opening of 1.05 mm and a bottom width of 170 μm. An example of a metabolite concentration gradient towards a respiring particle, in this case a murine blastocyst, in a conical hole is given in example 7.

Design J, shown in FIG. 5A. Metabolising particles on a detector plate. This design is the simplest possible diffusion compartment where the diffusion compartment is completely open and the metabolite gradient is recorded in two dimensions by a detector (5.3) embedded in the surface on which the metabolising particle (5.1) rests. 5B shows a cross section of the bottom at the level of the metabolising particle showing concentric iso-concentration lines (5.11) for the metabolite. 5C shows a hypothetical image (top or bottom view) as seen from the CCD camera (5.12), where the expected detector signal around each individual metabolising particle is visualized in grey tones. Micro-electrode profiles close to bovine embryos have been measured in this type of setup. This experiment is presented in example 3. The advantage of this design is its simplicity, the main disadvantage is the necessary accuracy in determining the local metabolite concentration in very close proximity to the metabolising particle. As spherical diffusion is extremely efficient over short distances spatial resolution is crucial.

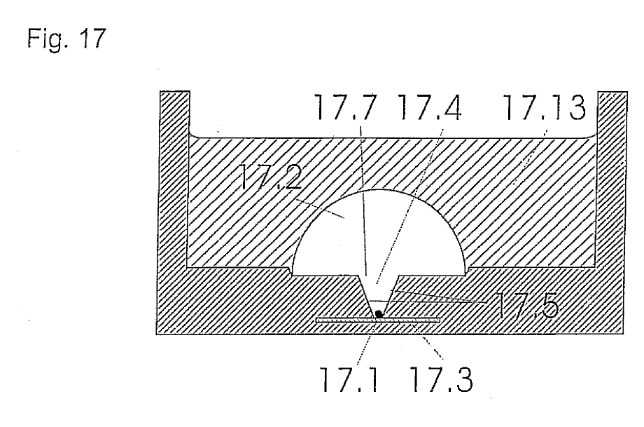

Design K, shown in FIG. 17. Plate with conical depressions. This design consists of an impermeable plate (17.5) with 500-3000 μm deep conical depressions of a suitable angle 30 (such as 15 to 60 degrees), placed in yet another slight depression with a hydrophilic surface. The remaining part of the plate surface is hydrophobic. A drop (17.2) of a suitable volume, 10-20 μl, fills the two depressions and constitutes the permeable diffusion barrier (17.4). The metabolising particle (17.1) is placed at the bottom of the conical depression (17.4) so that it is in contact with a disk of detector material (17.3) embedded in the impermeable material (17.3). The extension of the detector into a horizontal disc beneath the metabolising particle enable it to serve as a physical signal amplifier for an optical detection principle, as described above for design G. A layer of suitable oil above the drop prevents evaporation from the drop (coarse hatched liquid in vessel) and eliminates convection inside the drop such that the droplet of medium (17.2) for practical purposes is kept stagnant. Alternatively, the volume outside the conical depression (17.4) is part of the surrounding medium and thus not specifically included in the permeable diffusion barrier, unless it for other reasons remains stagnant. The permeability of the diffusion bather can be adjusted by moving the metabolising particle to other conical depressions (compartments) with different angles and/or depths, and the permeability of a particular conically shaped compartment can be calculated as explained in example 4 and the equations above.

Experimental results with such a design is presented in Example 7. The main advantage of this design is its simplicity, however to get measurable differences in metabolite concentration a fairly deep and narrow conical depression is required, thus approaching the simple bore described above. It is thus unclear if the conical depression brings an appreciable improvement in handling (especially with regard to removing the respiring objects from the device) when compared to a simple bore as described for device type A.

Irregular Systems which Constitute a Combination of the Above Designs, and where More Complex Modelling is Required to Describe the Concentration Gradient

Design L, shown in FIG. 8. Compartment with lid of adjustable thickness. This design employs a lid (8.4) as a non-liquid diffusion barrier composed of a material which is less permeable to the metabolite than the medium but still more permeable than the impermeable walls (8.5) of the compartment. The metabolising particle (8.1) is placed in a shallow well in an impermeable plate. A detector (8.3) is placed at the bottom of the well. The well has a metabolite permeable lid with varying thickness (8.4). We can thus adjust the diffusion barrier by covering the well with different sections of the lid with different thickness by simply displacing the lid horizontally. In this figure, the well and lid is covered with a droplet of medium (8.2), which is submerged under an oil cover (8.13) to prevent evaporation, but the medium could also fill the vessel without oil. The prime advantage of this design is the simplicity, ease of handling the metabolising particle in a relatively shallow well, and adjustable “compact” diffusion barrier with the lid. The main disadvantage is ensuring a tight fit between lid and the base plate. If the fit is not adequate, horizontal diffusion of metabolite into the compartment becomes possible.

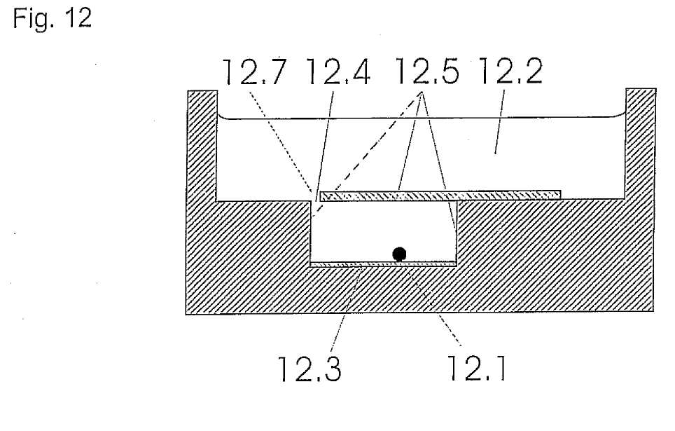

Design M, shown in FIG. 12. Compartment with partly open lid. This design is almost identical to the previous design, except that it employs an impermeable lid (12.5), which partly covers the well, leaving a small opening (12.7) as a diffusion barrier for the metabolite. The primer advantage of this design is the simplicity and adjustable lid to adjust the diffusion barrier, however a disadvantage is the expected difficulties in calibrating such an irregular system without keeping minutely track of the exact position of the lid in each measurement.

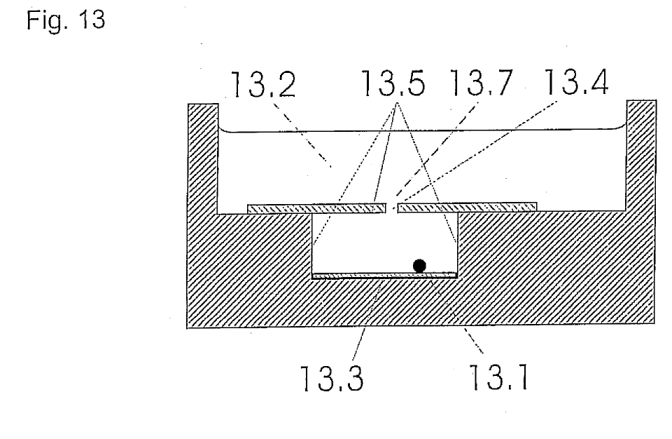

Design N, shown in FIG. 13 Compartment with impermeable lid with ventral pore. This design is functionally identical to the previous design, except that it employs a central pore (13.7) in the impermeable lid (13.5) as non-adjustable diffusion barrier. To change the diffusion barrier it is possible to exchange the lid for another with a larger pore size. It is very simple and probably easier to calibrate and use than the previous design. The main disadvantage is ensuring a tight fit between lid and the base plate. If the fit is not adequate, horizontal diffusion of metabolite into the compartment becomes possible.

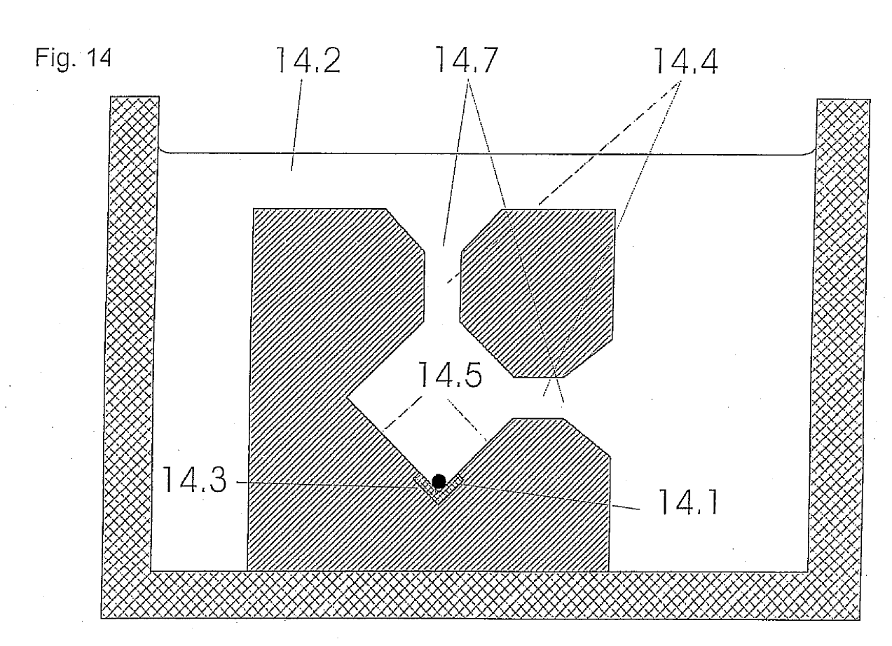

Design O, shown in FIG. 14 Cube with inlet and outlet for metabolising particle. This design is an impermeable cube (14.5) submerged in media (14.2) it contain two bores (14.4) connected to a central compartment. Each bore has a funnel shaped entrance (14.7). Initially the metabolising particle (14.1) is dropped into the vertically oriented funnel and allowed to settle by gravity onto the detector (14.3), the resulting metabolite concentration gradient is somewhat complex as metabolite is replenished through both bores. However numerical modelling can predict expected gradients given proper dimensions for the device. Once designed it can be calibrated by using metabolising particles with known metabolic rates. A clear advantage is the possibility to retrieve the metabolising particle by turning the cube and letting the particle fall out by gravity. The metabolising particle can also be deposited on other sides of the compartment with different detectors by turning the cube. A convective current can be forced through the cube if necessary to discharge the particle. A disadvantage is the more complicated design and possibility that the metabolising particle gets stuck inside the compartment.

### Example 6

### Optical Oxygen Measurement in Cylindrical Compartment with Murine Embryo

The general measuring principle was evaluated in a particular embodiment according to FIG. 11. The respiration activity of a mouse embryo at the blastocyst stage was measured according to the following description.

### Device

See detailed description in Example 5, version B, shown in FIG. 11.

It is composed of two impermeable pieces made of glass. A glass plate forms to bottom (11.5). Onto this plate is placed a small cylinder of glass (11.5) with a height, h of 4 mm. Through the centre of this class cylinder is a cylindrical hole (11.4) with a diameter, d, of 0.5 mm. At the end of the hole facing the vessel surface the hole is excavated (“hollowed out”) with a drill tip to form a small conical cavity into which the respiring particle (11.1) is placed. The upper walls of the conical cavity are covered with the oxygen quenchable porphyrin fluorophore (11.3) (Platinum (II)-octa-ethyl-porphyrin in polystyrene). The glass cylinder is affixed and sealed to the glass plate with dental wax, to avoid horizontal transport of oxygen at the interface between the two glass parts.

### Embryos:

Three to four-week-old immature female B6D2F1 mice (F1 hybrids between DBA/2J males and C57BL//6J females) were treated with 6 I.U. PMSG (Folligon® vet, Intervet, Denmark) i.p. on day 0. Two days later (day 3) they were treated with 6 I.U. Suigonan® Vet. (Suigonan, Intervet, Denmark 400 I.E. serumgonadotropin 200 I.E. choriongonadotropin). At the same day the females were mated to mature (fertility tested) male B6D2F1 mice. Two-cell embryos were flushed from the oviduct 2 days after mating (day 5) using medium M2 (Sigma Chemical, St. Louis USA). After flushing, the embryos were transferred to M16 (Sigma Chemical, St. Louis USA) medium and cultured at 37° C. under a 5% CO₂ in air atmosphere. Animals were kept in type II Macrolon cages (Techniplast, Italy) with free access to food (Altromin # 1314, Brogaarden, Denmark) and water.

A device according to FIG. 11 (design type B in Example 5) was placed in a Nunc 12 well dish in micro titer format (Nunc A/S, Roskilde Denmark), filled with M2 medium and left to equilibrate in the incubator for 60 minutes. One embryo at blastocyst stage (5 days after mating) was transferred to the device by ejecting it from the transfer pipette at the mouth of the central hole (11.7) and letting it sink to the bottom, into the chamber by gravity (11.1). The arrival of the embryo in the chamber was verified by inverted microscopy, allowing direct visual inspection of the chamber from below. The fluorescence intensity from the oxygen quenchable porphyrin fluorophor (Platinum (II)-octa-ethyl-porphyrin in polystyrene), in contact with the medium in the incubation chamber (11.3), was recorded using excitation light at 360 and 550 nm respectively and recording emission light at 650 nm in a Tecan. Spectraflour fluorescents plate reader. Fluorescence was recorded from 0 to 500 μs after excitation. Fluorescence intensities were converted to oxygen partial pressure using a modified Stern-Volmer equation, which adequately describes the response of most optrodes, according to Klimant et al 1995 (Fiber-optic oxygen microsensors, a new tool in aquatic biology. Limnol Oceanogr 40:1159-1165):

$$I = {I}_{0} [ α + ( 1 - α ) ( \frac{1}{1 + {K}_{sv} C} ) ]$$

Where α in the non-quenchable fraction of the fluorescence including scattered stray light, and I₀ is the fluorescence intensity in the absence after placing the embryo in the device, the oxygen partial pressure dropped from 21% (atmospheric concentration) to approximately 17% yielding a gradient of 4% oxygen (or 19% atmospheric saturation) over the height (4 mm) of the vertical cylindrical cavity of the device (11.4 in FIG. 11). The solubility of oxygen is 210 μM at 38° C. (incubation temperature) resulting in a gradient (dC/dX) of 100 μM cm⁻¹. The diffusion coefficient in growth media at 38° C. is approximately 3.45*10⁻⁵ cm² s⁻¹, which yields a flux of 3.45*10⁻¹² mol cm⁻² s⁻¹. As the tube has a cross section area of 0.00196 cm² this gives an embryo specific respiration rate of 0.677*10⁻¹⁴ mol embryo⁻¹ s⁻¹, or 0.546*10⁻⁹ l embryo⁻¹ h⁻¹ (0.546 nl O₂ h⁻¹).

### Example 7

### Microsensor Measurements in a Conical Depression

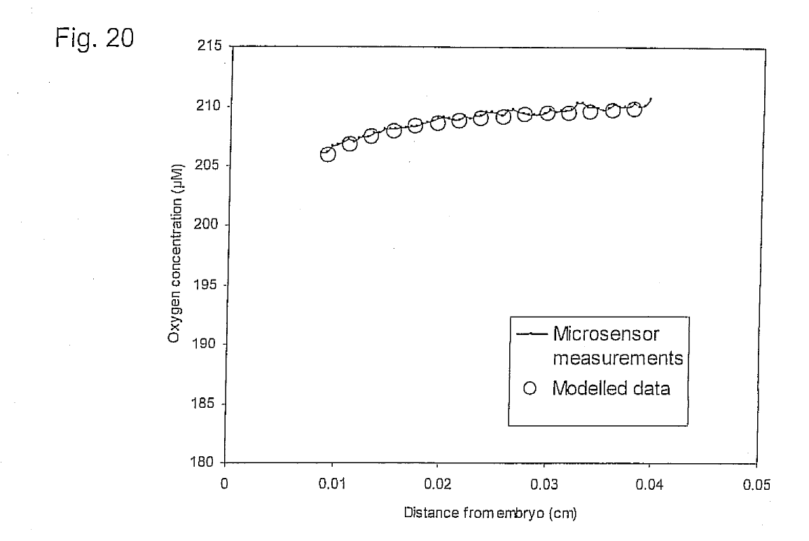

A 0.04 cm deep conical depression was created in the bottom of a 2 cm wide well in a polystyrene plastic plate by pressing a 60 degree pointed steel rod into its surface. A thin layer of dental wax was applied as a 4-mm diameter circle around the depression. Approx. 20 μl cultivation medium was pipetted into the depression and the area inside the wax circle, and a four day old approx. 100-μm diameter mouse embryo was placed in the bottom of the depression before 5 ml paraffin oil was poured into the well to cover the drop of medium. The plate was placed in a 37° C. water bath and the tip of an oxygen microsensor fixed in a motor-driven micromanipulator was positioned above the depression. A PC software which could control both the micromanipulator and acquire the signal from the microsensor amplifier was programmed to make oxygen measurements along a vertical line towards the embryo in steps of 5 micrometer. The measured concentrations versus microsensor distance to the embryo is shown in FIG. 20. At the embryo surface, which will be located approx. 0.015 cm from the tip/bottom of the depression, the concentration was 206 which can be translated to an oxygen consumption rate of 0.11 nl hour⁻¹ using formula 4.4.7. As can be seen from the figure, a complete concentration profile was measured towards the embryo, and using formula 4.4.7 to model this profile gives as very good fit, which confirms the validity of the model.

### REFERENCES

Hogan M C. Phosphorescence quenching method for measurement of intracellular PO2 in isolated skeletal muscle fibers.
J Appl Physiol. 1999 February; 86(2):720-4.
Trettnak W, Kolle C, Reininger F, Dolezal C, O'Leary P, Binot R A. Optical oxygen sensor instrumentation based on the detection of luminescence lifetime.
Adv Space Res. 1998; 22(10):1465-74.
Gewehr P M, Delpy D T. Optical oxygen sensor based on phosphorescence lifetime quenching and employing a polymer immobilised metalloporphyrin probe. Part 1. Theory and instrumentation. Med Biol Eng Comput. 1993 January; 31(1):2-10.
Klimant, I., Meyer, V., Kühl, M. 1995. Fiber-optic oxygen microsensors, a new tool in aquatic biology. Limnology and Oceanography, 40(6) 1159-1165
Glud, R. N., Ramsing, N. B., Gundersen, J. K., and Klimant, I. (1996). Planar optrodes, a new tool for fine scale measurements of two dimensional O2 distribution in benthic microbial communities. Marine Ecology Progress. Series 140: 217-226.
R N Glud, J K Gundersen, N B Ramsing (2000) Electrochemical and optical oxygen microsensors for in situ measurements. In in situ monitoring of aquatic systems: Chemical analysis and speciation. John Wiley & Sons Ltd (eds J Buffle & G Horvai). Chapter 2: 19-73

1. A device for non-invasive measurement of the individual metabolic rate of an individual substantially spherical metabolizing particle, which device comprises
a) at least one compartment, said at least one compartment being defined by a diffusion barrier and said at least one compartment being capable of retaining a medium with a substantially spherical metabolizing particle, said diffusion barrier is arranged around the substantially spherical metabolizing particle to restrict and reduce the diffusive flux of metabolites to and from the particle, allowing metabolite transport through the diffusion barrier to the substantially spherical metabolizing particle by means of diffusion wherein the medium within the compartment is stagnant so that the medium cannot mix by turbulent flow and so that a linear metabolite diffusion gradient is established from the substantially spherical metabolizing particle and throughout the medium in said at least one compartment,
b) at least one detector for measuring the concentration of a metabolite inside the compartment, the transverse dimension of said compartment being less than 1.5 millimeter, and the height of said compartment being larger than the transverse dimension of said compartment,
so that transport of metabolites to and/or from said particle through said medium in said at least one compartment occurs only through diffusion.

2. A non-invasive method for determining the metabolic rate of a substantially spherical metabolizing particle, comprising
a) providing at least one device as defined in claim 1,
b) arranging a substantially spherical metabolizing particle in the medium of a
compartment,
c) measuring a metabolite concentration inside the compartment obtaining a metabolite concentration measure, and
d) correlating said metabolite concentration measure to a metabolic rate of said substantially spherical metabolizing particle.

3. The method according to claim 2, wherein metabolite is supplied to the substantially spherical metabolizing particle by diffusion through the medium.

4. The method according to claim 2, wherein the substantially spherical metabolizing particle is cultured in the compartment.

5. The method according to claim 2, wherein the metabolic rate of said substantially spherical metabolizing particle is determined by determining a metabolite diffusion gradient in the compartment based on the measured metabolite concentration, and correlating said metabolite diffusion gradient to the metabolic rate of said substantially spherical metabolizing particle.

6. The method according to claim 2, wherein the metabolite concentration is a gas partial pressure.

7. The method according to claim 6, wherein the gas partial pressure is the partial pressure of oxygen or carbon dioxide.

8. The method according to claim 2, wherein the substantially spherical metabolizing particle is selected from the group consisting of an embryo, at least one cancer cell, at least one stem cell, embryonal stem cells, C. elegans and multicellular organisms.

9. A method for regulating metabolite supply to a substantially spherical metabolizing particle during culturing, comprising
a) providing at least one device comprising a compartment with a medium,
b) culturing a substantially spherical metabolizing particle in the medium of the
compartment,
c) measuring a metabolite concentration inside the compartment obtaining a metabolite concentration measure, and optionally
d) correlating said metabolite concentration measure to a metabolic rate of said substantially spherical metabolizing particle and optionally
e) regulating the metabolite supply depending on the metabolite concentration measure and/or the metabolic rate of said substantially spherical metabolizing particle.

10. The method according to claim 9, wherein at least one of the devices is as defined in claim 1.

11. The method according to claim 9, wherein the metabolite is oxygen and the metabolic process is respiration.

12. The method according to claim 9, wherein the regulation is conducted by changing the metabolite concentration outside the compartment.

13. The method according to claim 9, wherein the regulation is conducted by changing the dimensions of the compartment.

14. The method according to claim 9, wherein the regulation is conducted by changing the diffusion barrier of the compartment.

15. A method for selecting a viable embryo comprising,
a) determining the metabolic rate of the embryo at least once during culturing, and
b) selecting the embryo having an optimal metabolic rate.

16. The method according to claim 15, wherein the determination of the metabolic rate is conducted without causing any change in the growth conditions experienced by the embryo.

17. The method according to claim 15, wherein the metabolic rate is measured in a device as defined by claim 1.

18. A non-invasive method for determining the metabolic rate of a metabolizing particle, comprising
a) providing at least one device as defined in claim 1,
b) culturing a metabolizing particle in the medium of a compartment,
c) reducing metabolite supply to the medium during at least a part of the culturing
period,
d) measuring a metabolite concentration inside the compartment obtaining a metabolite concentration measure after the metabolite supply has been reduced, and
e) correlating said metabolite concentration measure to a metabolic rate of said substantially spherical metabolizing particle.

19. The method according to claim 18, wherein the metabolite is oxygen and the metabolic rate is the respiration rate.
